SEQUENCE LISTING (63 sequences)

SEQ ID NO: 1 - DNA, 21 bp, Artificial Sequence
  RNA/DNA, synthetic, RNA with 2′deoxythymidines at 3′ end
gugauguaugucagagagutt

SEQ ID NO: 2 - DNA, 21 bp, Artificial Sequence
  RNA/DNA, synthetic, RNA with 2′deoxythymidines at 3′ end
acucucugacauacaucactt

SEQ ID NO: 3 - DNA, 21 bp, Artificial Sequence
  RNA/DNA, synthetic, RNA with 2′deoxythymidines at 3′ end
gugauguaugucagagagutt

SEQ ID NO: 4 - DNA, 21 bp, Artificial Sequence
  RNA/DNA, synthetic, RNA with 2′deoxythymidines at 3′ end
acucucugacauacaucactt

SEQ ID NO: 5 - DNA, 21 bp, Artificial Sequence
  RNA/DNA, synthetic, RNA with 2′deoxythymidines at 3′ end
acucucugacauacaucactt

SEQ ID NO: 6 - DNA, 21 bp, Artificial Sequence
  RNA/DNA, synthetic, RNA with 2′deoxythymidines at 3′ end
acucucugacauacaucactt

SEQ ID NO: 7 - DNA, 21 bp, Artificial Sequence
  RNA/DNA, synthetic, RNA with 2′deoxythymidines at 3′ end
acucucugacauacaucactt

SEQ ID NO: 8 - DNA, 21 bp, Artificial Sequence
  RNA/DNA, synthetic, RNA with 2′deoxythymidines at 3′ end
gugauguaugucagagagutt

SEQ ID NO: 9 - DNA, 20 bp, Artificial Sequence
  RNA/DNA, synthetic, RNA with 2′deoxythymidines at 3′ end
acucucugacauacaucctt

SEQ ID NO: 10 - DNA, 21 bp, Artificial Sequence
  RNA/DNA, synthetic, RNA with 2′deoxythymidines at 3′ end
gugauguaugucagagagutt

SEQ ID NO: 11 - DNA, 21 bp, Artificial Sequence
  RNA/DNA, synthetic, RNA with 2′deoxythymidines at 3′ end
acucucugacauacaucactt

SEQ ID NO: 12 - DNA, 21 bp, Artificial Sequence
  RNA/DNA, synthetic, RNA with 2′deoxythymidines at 3′ end
acucucugacauacaucactt

SEQ ID NO: 13 - DNA, 21 bp, Artificial Sequence
  RNA/DNA, synthetic, RNA with 2′deoxythymidines at 3′ end
gugauguaugucagagagutt

SEQ ID NO: 14 - DNA, 21 bp, Artificial Sequence
  RNA/DNA, synthetic, RNA with 2′deoxythymidines at 3′ end
acucucugacauacaucactt

SEQ ID NO: 15 - DNA, 21 bp, Artificial Sequence
  RNA/DNA, synthetic, RNA with 2′deoxythymidines at 3′ end
gugauguaugucagagagutt

SEQ ID NO: 16 - DNA, 21 bp, Artificial Sequence
  RNA/DNA, synthetic, RNA with 2′deoxythymidines at 3′ end
acucucugacauacaucactt

SEQ ID NO: 17 - DNA, 21 bp, Artificial Sequence
  RNA/DNA, synthetic, RNA with 2′deoxythymidines at 3′ end
ugguguuuggcaaaguucutt

SEQ ID NO: 18 - DNA, 21 bp, Artificial Sequence
  RNA/DNA, synthetic, RNA with 2′deoxythymidines at 3′ end
agaacuuugccaaacaccatt

SEQ ID NO: 19 - DNA, 21 bp, Artificial Sequence
  RNA/DNA, synthetic, RNA with 2′deoxythymidines at 3′ end
ugguguuuggcaaaguucutt

SEQ ID NO: 20 - DNA, 21 bp, Artificial Sequence
  RNA/DNA, synthetic, RNA with 2′deoxythymidines at 3′ end
agaacuuugccaaacaccatt

SEQ ID NO: 21 - DNA, 21 bp, Artificial Sequence
  RNA/DNA, synthetic, RNA with 2′deoxythymidines at 3′ end
agaacuuugccaaacaccatt

SEQ ID NO: 22 - DNA, 21 bp, Artificial Sequence
  RNA/DNA, synthetic, RNA with 2′deoxythymidines at 3′ end
agaacuuugccaaacaccatt

SEQ ID NO: 23 - DNA, 21 bp, Artificial Sequence
  RNA/DNA, synthetic, RNA with 2′deoxythymidines at 3′ end
agaacuuugccaaacaccatt

SEQ ID NO: 24 - DNA, 21 bp, Artificial Sequence
  RNA/DNA, synthetic, RNA with 2′deoxythymidines at 3′ end
gauuauguccgguuauguatt

SEQ ID NO: 25 - DNA, 21 bp, Artificial Sequence
  RNA/DNA, synthetic, RNA with 2′deoxythymidines at 3′ end
uacauaaccggacauaauctt

SEQ ID NO: 26 - DNA, 21 bp, Artificial Sequence
  RNA/DNA, synthetic, RNA with 2′deoxythymidines at 3′ end
gauuauguccgguuauguatt

SEQ ID NO: 27 - DNA, 21 bp, Artificial Sequence
  RNA/DNA, synthetic, RNA with 2′deoxythymidines at 3′ end
gauuauguccgguuauguatt

SEQ ID NO: 28 - DNA, 21 bp, Artificial Sequence
  RNA/DNA, synthetic, RNA with 2′deoxythymidines at 3′ end
gauuauguccgguuauguatt

SEQ ID NO: 29 - DNA, 21 bp, Artificial Sequence
  RNA/DNA, synthetic, RNA with 2′deoxythymidines at 3′ end
gauuauguccgguuauguatt

SEQ ID NO: 30 - DNA, 21 bp, Artificial Sequence
  RNA/DNA, synthetic, RNA with 2′deoxythymidines at 3′ end
gauuauguccgguuauguatt

SEQ ID NO: 31 - DNA, 21 bp, Artificial Sequence
  RNA/DNA, synthetic, RNA with 2′deoxythymidines at 3′ end
gauuauguccgguuauguatt

SEQ ID NO: 32 - DNA, 21 bp, Artificial Sequence
  RNA/DNA, synthetic, RNA with 2′deoxythymidines at 3′ end
gauuauguccgguuauguatt

SEQ ID NO: 33 - DNA, 21 bp, Artificial Sequence
  RNA/DNA, synthetic, RNA with 2′deoxythymidines at 3′ end
gauuauguccgguuauguatt

SEQ ID NO: 34 - DNA, 21 bp, Artificial Sequence
  RNA/DNA, synthetic, RNA with 2′deoxythymidines at 3′ end
gauuauguccgguuauguatt

SEQ ID NO: 35 - DNA, 21 bp, Artificial Sequence
  RNA/DNA, synthetic, RNA with 2′deoxythymidines at 3′ end
gauuauguccgguuauguatt

SEQ ID NO: 36 - DNA, 21 bp, Artificial Sequence
  RNA/DNA, synthetic, RNA with 2′deoxythymidines at 3′ end
gauuauguccgguuauguatt

SEQ ID NO: 37 - DNA, 21 bp, Artificial Sequence
  RNA/DNA, synthetic, RNA with 2′deoxythymidines at 3′ end
gauuauguccgguuauguatt

SEQ ID NO: 38 - DNA, 21 bp, Artificial Sequence
  RNA/DNA, synthetic, RNA with 2′deoxythymidines at 3′ end
gauuauguccgguuauguatt

SEQ ID NO: 39 - DNA, 21 bp, Artificial Sequence
  RNA/DNA, synthetic, RNA with 2′deoxythymidines at 3′ end
gauuauguccgguuauguatt

SEQ ID NO: 40 - DNA, 21 bp, Artificial Sequence
  RNA/DNA, synthetic, RNA with 2′deoxythymidines at 3′ end
gauuauguccgguuauguatt

SEQ ID NO: 41 - DNA, 21 bp, Artificial Sequence
  RNA/DNA, synthetic, RNA with 2′deoxythymidines at 3′ end
gauuauguccgguuauguatt

SEQ ID NO: 42 - DNA, 21 bp, Artificial Sequence
  RNA/DNA, synthetic, RNA with 2′deoxythymidines at 3′ end
gauuauguccgguuauguatt

SEQ ID NO: 43 - DNA, 21 bp, Artificial Sequence
  RNA/DNA, synthetic, RNA with 2′deoxythymidines at 3′ end
gauuauguccgguuauguatt

SEQ ID NO: 44 - DNA, 21 bp, Artificial Sequence
  RNA/DNA, synthetic, RNA with 2′deoxythymidines at 3′ end
gauuauguccgguuauguatt

SEQ ID NO: 45 - DNA, 21 bp, Artificial Sequence
  RNA/DNA, synthetic, RNA with 2′deoxythymidines at 3′ end
uacauaaccggacauaauctt

SEQ ID NO: 46 - DNA, 21 bp, Artificial Sequence
  RNA/DNA, synthetic, RNA with 2′deoxythymidines at 3′ end
uacauaaccggacauaauctt

SEQ ID NO: 47 - DNA, 21 bp, Artificial Sequence
  RNA/DNA, synthetic, RNA with 2′deoxythymidines at 3′ end
uacauaaccggacauaauctt

SEQ ID NO: 48 - DNA, 21 bp, Artificial Sequence
  RNA/DNA, synthetic, RNA with 2′deoxythymidines at 3′ end
uacauaaccggacauaauctt

SEQ ID NO: 49 - DNA, 21 bp, Artificial Sequence
  RNA/DNA, synthetic, RNA with 2′deoxythymidines at 3′ end
uacauaaccggacauaauctt

SEQ ID NO: 50 - DNA, 21 bp, Artificial Sequence
  RNA/DNA, synthetic, RNA with 2′deoxythymidines at 3′ end
uacauaaccggacauaauctt

SEQ ID NO: 51 - DNA, 21 bp, Artificial Sequence
  RNA/DNA, synthetic, RNA with 2′deoxythymidines at 3′ end
uacauaaccggacauaauctt

SEQ ID NO: 52 - DNA, 21 bp, Artificial Sequence
  RNA/DNA, synthetic, RNA with 2′deoxythymidines at 3′ end
uacauaaccggacauaauctt

SEQ ID NO: 53 - DNA, 21 bp, Artificial Sequence
  RNA/DNA, synthetic, RNA with 2′deoxythymidines at 3′ end
uacauaaccggacauaauctt

SEQ ID NO: 54 - DNA, 21 bp, Artificial Sequence
  RNA/DNA, synthetic, RNA with 2′deoxythymidines at 3′ end
uacauaaccggacauaauctt

SEQ ID NO: 55 - DNA, 21 bp, Artificial Sequence
  RNA/DNA, synthetic, RNA with 2′deoxythymidines at 3′ end
uacauaaccggacauaauctt

SEQ ID NO: 56 - DNA, 21 bp, Artificial Sequence
  RNA/DNA, synthetic, RNA with 2′deoxythymidines at 3′ end
uacauaaccggacauaauctt

SEQ ID NO: 57 - DNA, 21 bp, Artificial Sequence
  RNA/DNA, synthetic, RNA with 2′deoxythymidines at 3′ end
uacauaaccggacauaauctt

SEQ ID NO: 58 - DNA, 21 bp, Artificial Sequence
  RNA/DNA, synthetic, RNA with 2′deoxythymidines at 3′ end
uacauaaccggacauaauctt

SEQ ID NO: 59 - DNA, 21 bp, Artificial Sequence
  RNA/DNA, synthetic, RNA with 2′deoxythymidines at 3′ end
uacauaaccggacauaauctt

SEQ ID NO: 60 - DNA, 21 bp, Artificial Sequence
  RNA/DNA, synthetic, RNA with 2′deoxythymidines at 3′ end
uacauaaccggacauaauctt

SEQ ID NO: 61 - DNA, 21 bp, Artificial Sequence
  RNA/DNA, synthetic, RNA with 2′deoxythymidines at 3′ end
uacauaaccggacauaauctt

SEQ ID NO: 62 - DNA, 21 bp, Artificial Sequence
  RNA/DNA, synthetic, RNA with 2′deoxythymidines at 3′ end
uacauaaccggacauaauctt

SEQ ID NO: 63 - DNA, 21 bp, Artificial Sequence
  RNA/DNA, synthetic, RNA with 2′deoxythymidines at 3′ end
uacauaaccggacauaauctt

(19) United States
(12) Patent Application Publication
(10) Pub. No.: US 2004/0198640 A1
(43) Pub. Date: Oct. 7, 2004

(54) Stabilized polynucleotides for use in RNA interference

(75) Inventors: Devin Leake, Denver, CO (US); Angela Reynolds, Conifer, CO (US); Anastasia Khvorova, Boulder, CO (US); William Marshall, Boulder, CO (US); Stephen Scaringe, Lafayette, CO (US)

Correspondence Address: KALOW & SPRINGUT LLP, 488 MADISON AVENUE, 19TH FLOOR, NEW YORK, NY, 10022, US

(73) Assignee: Dharmacon, Inc., Lafayette, CO (US)

(21) Appl. No.: 10/406,908

(22) Filed: Apr. 2, 2003

(51) Int. Cl. A61K 48/00

(52) U.S. Cl. 514/8; 514/44

(57) ABSTRACT

Methods and compositions for performing RNA interference comprising a wide variety of stabilized polynucleotides suitable for use in serum-containing media and for in vivo applications, such as therapeutic applications, are provided. These polynucleotides permit effective and efficient applications of RNA interference to applications such as diagnostics and therapeutics through the use of one or more modifications including orthoesters, terminal conjugates, modified linkages and 2′modified nucleotides.

### FIELD OF THE INVENTION

The present invention relates to the field of stabilized polynucleotides.

### BACKGROUND

Relatively recent discoveries in the field of RNA metabolism have revealed that the uptake of double stranded RNA (dsRNA) can induce a phenomenon known as RNA interference (RNAi). RNAi is a process by which a polynucleotide inhibits the activity of another nucleotide sequence, such as messenger RNA. This phenomenon has been observed in cells of a diverse group of organisms, including humans, suggesting its promise as a novel therapeutic approach to the genetic control of human disease.

In most organisms, RNAi is effective when using relatively long dsRNA. Unfortunately, in mammalian cells, the use of long dsRNA to induce RNAi has been met with only limited success. In large part, this ineffectiveness is due to induction of the interferon response, which results in a general, as opposed to targeted, inhibition of protein synthesis.

Recently, it has been shown that when short RNA duplexes are introduced into mammalian cells in culture, sequence-specific inhibition of target mRNA can be realized without inducing an interferon response. These short dsRNAs, referred to as small interfering RNAs (siRNAs), can act catalytically at sub-molar concentrations to cleave greater than 95% of the target mRNA in a cell. A description of the mechanisms for siRNA activity, as well as some of its applications is described in Provost et al., Ribonuclease Activity and RNA Binding of Recombinant Human Dicer, E.M.B.O.J., 2002 Nov., 1, 21(21): 5864-5874; Tabara et al., The dsRNA Binding Protein RDE-4 Interacts with RDE-1, DCR-1 and a DexH-box Helicase to Direct RNAi in C. elegans, Cell. Jun. 28, 2002, 109(7):861-71; Ketting et al., Dicer Functions in RNA Interference and in Synthesis of Small RNA Involved in Developmental Timing in C. elegans; and Martinez et al., Single-Stranded Antisense siRNAs Guide Target RNA Cleavage in RNAi, Cell Sep. 6, 2002, 110(5):563, all of which are incorporated by reference herein.

RNA-induced gene silencing in mammalian cells is presently believed to implicate at least three different levels of control: (i) transcription inactivation (siRNA-guided DNA and histone methylation); (ii) siRNA-induced mRNA degradation; and (iii) mRNA-induced transcriptional attenuation. The interference effect can be long lasting and can be detected after many cell divisions. Consequently, the ability to assess gene function via siRNA mediated methods, as well as to develop therapies for over-expressed genes, represents an exciting and valuable tool that will accelerate genome-wide investigations across a broad range of biomedical and biological research.

Unfortunately, when naked siRNA molecules are introduced into blood, serum, or serum-containing media, they are nearly immediately degraded. This degradation is due in part to the presence of nucleases and other substances that reduce or eliminate the effectiveness of polynucleotides. Consequently, the use of naked siRNA in cell culture, animal studies, and studies aimed at developing therapeutics, has limited potential benefits.

Some progress has been made in other applications toward developing modified ribonucleic acids that exhibit improved stability under the above-described conditions, while retaining biological functionality. For example, literature related to ribonucleic acid technologies such as ribozyme stabilization and long antisense DNA stabilization suggest that partial modification of the sugar ring, or the backbone of an RNA molecule, could improve its stability so that complete degradation in blood, serum, or serum-containing media would be prevented, while maintaining some of the nucleic acid's functionality. Known modifications for these applications include, for example, fluoro, 2′-O-methyl, amine and deoxy modifications at the 2′ position of the sugar ring.

However, to date there has been only limited focus on the use and optimization of these and other modifications in connection with RNAi. One limitation on the use of known modifications is that although they increase stability, this benefit comes at a price. For example, some modifications decrease functionality, thereby requiring higher effective doses; others eliminate functionality entirely, and still others are toxic.

Thus, there remains a need to develop compositions and methods of using functional stabilized polynucleotides that retain potency. The present invention offers a solution.

### SUMMARY OF THE INVENTION

The present invention is directed to compositions and methods for performing RNA interference. The compositions and methods of the present invention allow for performing RNA interference with stabilized, functional double stranded polynucleotides. They are particularly advantageous for use in applications that require exposure to blood, serum, serum-containing media, and other biological material that contains nucleases or other factors that tend to degrade nucleic acids.

According to a first embodiment, the present invention provides a double stranded polynucleotide having a sense strand comprising a polynucleotide comprised of at least one orthoester modified nucleotide, and an antisense strand comprising a polynucleotide comprised of at least one 2′ modified nucleotide unit.

According to a second embodiment, the present invention provides a double stranded polynucleotide having a sense strand comprising a polynucleotide comprised of at least one orthoester modified nucleotide, an antisense strand comprising a polynucleotide comprised of at least one 2′ modified nucleotide, and a conjugate.

According to a third embodiment, the present invention provides a double stranded polynucleotide having a sense strand comprising at least one orthoester modified nucleotide, an antisense strand, and a conjugate.

According to a fourth embodiment, the present invention provides a double stranded polynucleotide having a sense strand, an antisense strand, and a conjugate, wherein the sense strand and/or the antisense strand have at least one 2′ modified nucleotide.

According to a fifth embodiment, the present invention provides a double stranded polyribonucleotide having a sense strand comprising at least one orthoester modified nucleotide, an antisense strand comprising at least one 2′ modified nucleotide selected from the group consisting of a 2′ halogen modified nucleotide, a 2′ amine modified nucleotide, a 2′-O-alkyl modified nucleotide, and a 2′ alkyl modified nucleotide, and a conjugate selected from the group consisting of amino acids, peptides, polypeptides, proteins, sugars, carbohydrates, lipids, polymers, nucleotides, polynucleotides, and combinations thereof, wherein the polyribonucleotide comprises between 18 and 30 nucleotide base pairs.

According to a sixth embodiment, the present invention provides a composition comprising one of the structures below:

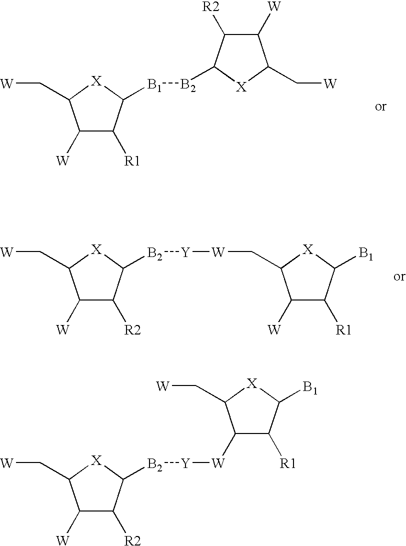

wherein each of B₁ and B₂ is a nitrogenous base, carbocycle, or heterocycle; X is selected from the group consisting of O, S, C, and N; W is selected from the group consisting of an OH, a phosphate, a phosphate ester, a phosphodiester, a phosphotriester, a modified internucleotide linkage, a conjugate, a nucleotide, and a polynucleotide; R1 is an orthoester; R2 is selected from the group consisting of a 2′-O-alkyl group, an alkyl group, an amine and a halogen; and Y is a nucleotide or polynucleotide. The dashed lines between B₁ and B₂ indicate interaction by hydrogen bonding between nitrogenous bases.

According to a seventh embodiment, the present invention provides a method of performing RNA interference. This method is comprised of exposing a double stranded polynucleotide to a target nucleic acid. The double stranded polynucleotide is comprised of a sense strand and an antisense strand, and at least one of said sense strand and said antisense strand comprises at least one orthoester modified nucleotide.

According to an eighth embodiment, the present invention provides another method of performing RNA interference. This method is comprised of exposing a double stranded polynucleotide to a target nucleic acid, wherein the double stranded polynucleotide is comprised of a sense strand, an antisense strand, and a conjugate. According to this embodiment, either the sense strand or the antisense strand comprises a 2′ modified nucleotide.

The compositions of the present invention can render double stranded polynucleotides resistant to nuclease degradation, while maintaining biological functionality. By for example, using double stranded polynucleotides with at least one orthoester modified nucleotide, such as on the sense strand, and at least one other modification, such as at an appropriate position on the antisense strand, one can enhance stability while retaining functionality in RNA interference applications. Additionally, using double stranded polynucleotides with one or more 2′ modifications, and/or modified internucleotide linkages, in conjunction with conjugates, in RNA interference applications, can also provide enhanced stability while retaining functionality, even in the absence of an orthoester modification on either strand.

For a better understanding of the present invention together with other and further advantages and embodiments, reference is made to the following description taken in conjunction with the examples, the scope of the which is set forth in the appended claims.

### BRIEF DESCRIPTION OF THE FIGURES

The preferred embodiments of the present invention have been chosen for purposes of illustration and description but are not intended to restrict the scope of the invention in any way. The benefits of the preferred embodiments of certain aspects of the invention are shown in the accompanying figures, wherein:

FIG. 1A illustrates the functionality of orthoester modifications on sense and/or antisense strands as measured 24 hours post-transfection.

FIG. 1B illustrates the functionality of orthoester modifications on sense and/or antisense strands as measured 48 hours post-transfection.

FIG. 2A illustrates the functionality of orthoester modifications on sense and/or antisense strands in conjunction with other modifications, as measured 24 hours post-transfection.

FIG. 2B illustrates the functionality of orthoester modifications on sense and/or antisense strands in conjunction with other modifications, as measured 72 hours post-transfection.

FIG. 2C illustrates the functionality of orthoester modifications on sense and/or antisense strands in conjunction with other modifications as measured 144 hours post-transfection.

FIG. 3 illustrates the effects of modifications on an antisense strand in an siRNA.

FIG. 4 illustrates the effects of modifications on a sense strand in an siRNA.

FIG. 5 illustrates the effects of thio-based modifications of an antisense strand.

FIG. 6 illustrates the effects of phosphorothioate modifications in both sense and antisense strands.

FIG. 7 illustrates the effects of 2′-O-methyl modifications in both sense and antisense strands.

FIG. 8 illustrates the effects of siRNAs that are 2′-deoxy-RNA hybrids.

FIG. 9 illustrates the functionality of a cholesterol conjugate at the 5′ end of a sense strand.

FIG. 10 illustrates the functionality of a PEG conjugate at the 5′ end of a sense strand.

FIG. 11 illustrates the reduction in functional dose of a modified siRNA having a cholesterol conjugate at the 5′ end of a sense strand.

FIG. 12 illustrates protected RNA nucleoside phosphoramidites that can be used for Dharmacon 2′-ACE RNA synthesis chemistry.

FIG. 13 illustrates an outline of a Dharmacon RNA synthesis cycle.

FIG. 14 Illustrates the structure of a preferred 2′-ACE protected RNA immediately prior to 2′-deprotection.

FIG. 15A illustrates functionality consequences of a single 2′-deoxy modification on an otherwise naked double stranded polyribonucleotide.

FIG. 15B illustrates functionality consequences of two tandem 2′-deoxy modifications on an otherwise naked double stranded polyribonucleotide.

FIG. 15C illustrates functionality consequences of three tandem 2′-deoxy modifications on an otherwise naked double stranded polyribonucleotide.

FIG. 16A illustrates functionality consequences of a single 2′-O-methyl modification throughout an otherwise naked double stranded polyribonucleotide.

FIG. 16B illustrates functionality consequences of two tandem 2′-O-methyl modifications throughout an otherwise naked double stranded polyribonucleotide.

FIG. 16C illustrates functionality consequences of three tandem 2′-O-methyl modifications throughout an otherwise naked double stranded polyribonucleotide.

FIG. 17 illustrates functionality consequences of modifications in the sense and the antisense strands.

FIG. 18 illustrates the effect of a conjugate comprising a 5′ cholesterol moiety on passive uptake of double stranded polyribonucleotides.

### DETAILED DESCRIPTION

The present invention will now be described in connection with preferred embodiments. These embodiments are presented to aid in an understanding of the present invention and are not intended, and should not be construed, to limit the invention in any way. All alternatives, modifications and equivalents that may become apparent to those of ordinary skill upon reading this disclosure are included within the spirit and scope of the present invention.

This disclosure is not a primer on compositions and methods for performing RNA interference. Basic concepts known to those skilled in the art have not been set forth in detail.

The present invention is directed to compositions and methods for performing RNA interference, including siRNA-induced gene silencing. Through the use of the present invention, modified polynucleotides, and derivatives thereof, one may improve the efficiency of RNA interference applications.

Unless stated otherwise, the following terms and phrases have the meanings provided below:

Alkyl

The term “alkyl” refers to a hydrocarbyl moiety that can be saturated or unsaturated, and substituted or unsubstituted. It may comprise moieties that are linear, branched, cyclic and/or heterocyclic, and contain functional groups such as ethers, ketones, aldehydes, carboxylates, etc.

Exemplary alkyl groups include but are not limited to substituted and unsubstituted groups of methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl and alkyl groups of higher number of carbons, as well as 2-methylpropyl, 2-methyl-4-ethylbutyl, 2,4-diethylpropyl, 3-propylbutyl, 2,8-dibutyldecyl, 6,6-dimethyloctyl, 6-propyl-6-butyloctyl, 2-methylbutyl, 2-methylpentyl, 3-methylpentyl, and 2-ethylhexyl. The term alkyl also encompasses alkenyl groups, such as vinyl, allyl, aralkyl and alkynyl groups.

Substitutions within an alkyl group can include any atom or group that can be tolerated in the alkyl moiety, including but not limited to halogens, sulfurs, thiols, thioethers, thioesters, amines (primary, secondary, or tertiary), amides, ethers, esters, alcohols and oxygen. The alkyl groups can by way of example also comprise modifications such as azo groups, keto groups, aldehyde groups, carboxyl groups, nitro, nitroso or nitrile groups, heterocycles such as imidazole, hydrazino or hydroxylamino groups, isocyanate or cyanate groups, and sulfur containing groups such as sulfoxide, sulfone, sulfide, and disulfide.

Further, alkyl groups may also contain hetero substitutions, which are substitutions of carbon atoms, by for example, nitrogen, oxygen or sulfur. Heterocyclic substitutions refer to alkyl rings having one or more heteroatoms. Examples of heterocyclic moieties include but are not limited to morpholino, imidazole, and pyrrolidino.

2′-O-Alkyl Modified Nucleotide

The phrase “2′-O-alkyl modified nucleotide” refers to a nucleotide unit having a sugar moiety, for example a deoxyribosyl moiety that is modified at the 2′ position such that an oxygen atom is attached both to the carbon atom located at the 2′ position of the sugar and to an alkyl group.

Amine and 2′ Amine Modified Nucleotide

The term “amine” refers to moieties that can be derived directly or indirectly from ammonia by replacing one, two, or three hydrogen atoms by other groups, such as, for example, alkyl groups. Primary amines have the general structures RNH₂ and secondary amines have the general structure R₂NH. The phrase “2′ amine modified nucleotide” refers to a nucleotide unit having a sugar moiety that is modified with an amine or nitrogen containing group attached to the 2′ position of the sugar.

The term amine includes, but is not limited to methylamine, ethylamine, propylamine, isopropylamine, aniline, cyclohexylamine, benzylamine, polycyclic amines, heteroatom substituted aryl and alkylamines, dimethylamine, diethylamine, diisopropylamine, dibutylamine, methylpropylamine, methylhexylamine, methylcyclopropylamine, ethylcylohexylamine, methylbenzylamine, methycyclohexylmethylamine, butylcyclohexylamine, morpholine, thiomorpholine, pyrrolidine, piperidine, 2,6-dimethylpiperidine, piperazine, and heteroatom substituted alkyl or aryl secondary amines.

Antisense Strand

The phrase “antisense strand” as used herein, refers to a polynucleotide that is substantially or 100% complementary, to a target nucleic acid of interest. An antisense strand may be comprised of a polynucleotide that is RNA, DNA or chimeric RNA/DNA. For example, an antisense strand may be complementary, in whole or in part, to a molecule of messenger RNA, an RNA sequence that is not mRNA (e.g., tRNA, rRNA and hnRNA) or a sequence of DNA that is either coding or non-coding.

Complementary

The term “complementary” refers to the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of stable duplexes.

Perfect complementarity or 100% complementarity refers to the situation in which each nucleotide unit of one polynucleotide strand can hydrogen bond with each nucleotide unit of a second polynucleotide strand. Less than perfect complementarity refers to the situation in which some, but not all, nucleotide units of two strands can hydrogen bond with each other. For example, for two 20-mers, if only two base pairs on each strand can hydrogen bond with each other, the polynucleotide strands exhibit 10% complementarity. In the same example, if 18 base pairs on each strand can hydrogen bond with each other, the polynucleotide strands exhibit 90% complementarity. Substantial complementarity refers to polynucleotide strands exhibiting 90% or greater complementarity.

Conjugate and Terminal Conjugate

The term “conjugate” refers to a molecule or moiety that alters the physical properties of a polynucleotide such as those that increase stability and/or facilitate uptake of double stranded RNA by itself. A “terminal conjugate” may be attached directly or through a linker to the 3′ and/or 5′ end of a polynucleotide or double stranded polynucleotide. An internal conjugate may be attached directly or indirectly through a linker to a base, to the 2′ position of the ribose, or to other positions that do not interfere with Watson-Crick base pairing, for example, 5-aminoallyl uridine.

In a double stranded polynucleotide, one or both 5′ ends of the strands of polynucleotides comprising the double stranded polynucleotide can bear a conjugate, and/or one or both 3′ ends of the strands of polynucleotides comprising the double stranded polynucleotide can bear a conjugate.

Conjugates may, for example, be amino acids, peptides, polypeptides, proteins, antibodies, antigens, toxins, hormones, lipids, nucleotides, nucleosides, sugars, carbohydrates, polymers such as polyethylene glycol and polypropylene glycol, as well as analogs or derivatives of all of these classes of substances. Additional examples of conjugates also include steroids, such as cholesterol, phospholipids, di- and tri-acylglycerols, fatty acids, hydrocarbons that may or may not contain unsaturation or substitutions, enzyme substrates, biotin, digoxigenin, and polysaccharides. Still other examples include thioethers such as hexyl-S-tritylthiol, thiocholesterol, acyl chains such as dodecandiol or undecyl groups, phospholipids such as di-hexadecyl-rac-glycerol, triethylammonium 1,2-di-O-hexadecyl-rac-glycero-3-H-phosphonate, polyamines, polyethylene glycol, adamantane acetic acid, palmityl moieties, octadecylamine moieties, hexylaminocarbonyl-oxycholesterol, farnesyl, geranyl and geranylgeranyl moieties.

Conjugates can also be detectable labels. For example, conjugates can be fluorophores. Conjugates can include fluorophores such as TAMRA, BODIPY, Cyanine derivatives such as Cy3 or Cy5 Dabsyl, or any other suitable fluorophore known in the art.

A conjugate may be attached to any position on the terminal nucleotide that is convenient and that does not substantially interfere with the desired activity of the polynucleotide(s) that bear it, for example the 3′ or 5′ position of a ribosyl sugar. A conjugate substantially interferes with the desired activity of an siRNA if it adversely affects its functionality such that the ability of the siRNA to mediate RNA interference is reduced by greater than 80% in an in vitro assay employing cultured cells, where the functionality is measured at 24 hours post transfection.

Deoxynucleotide

The term “deoxynucleotide” refers to a nucleotide or polynucleotide lacking an OH group at the 2′ or 3′ position of a sugar moiety with appropriate bonding and/or 2′,3′ terminal dideoxy, instead having a hydrogen bonded to the 2′ and/or 3′ carbon.

Deoxyribonucleotide

The terms “deoxyribonucleotide” and “DNA” refer to a nucleotide or polynucleotide comprising at least one ribosyl moiety that has an H at its 2′ position of a ribosyl moiety.

Functional Dose

A “functional dose” refers to a dose of siRNA that will be effective at causing a greater than or equal to 95% reduction in mRNA at levels of 100 nM at 24, 48, 72, and 96 hours following administration, while a “marginally functional dose” of siRNA will be effective at causing a greater than or equal to 50% reduction of mRNA at 100 nM at 24 hours following administration and a “non-functional dose” of RNA will cause a less than 50% reduction in mRNA levels at 100 nM at 24 hours following administration.

Halogen

The term “halogen” refers to an atom of either fluorine, chlorine, bromine, iodine or astatine. The phrase “2′ halogen modified nucleotide” refers to a nucleotide unit having a sugar moiety that is modified with a halogen at the 2′ position, attached directly to the 2′ carbon.

Internucleotide Linkage

The phrase “internucleotide linkage” refers to the type of bond or link that is present between two nucleotide units in a polynucleotide and may be modified or unmodified. The phrase “modified internucleotide linkage” includes all modified internucleotide linkages now known in the art or that come to be known and that, from reading this disclosure, one skilled in the art will conclude is useful in connection with the present invention. Internucleotide linkages may have associated counterions, and the term is meant to include such counterions and any coordination complexes that can form at the internucleotide linkages.

Modifications of internucleotide linkages include, but are not limited to, phosphorothioates, phosphorodithioates, methylphosphonates, 5′-alkylenephosphonates, 5′-methylphosphonate, 3′-alkylene phosphonates, borontrifluoridates, borano phosphate esters and selenophosphates of 3′-5′ linkage or 2′-5′ linkage, phosphotriesters, thionoalkylphosphotriesters, hydrogen phosphonate linkages, alkyl phosphonates, alkylphosphonothioates, arylphosphonothioates, phosphoroselenoates, phosphorodiselenoates, phosphinates, phosphoramidates, 3′-alkylphosphoramidates, aminoalkylphosphoramidates, thionophosphoramidates, phosphoropiperazidates, phosphoroanilothioates, phosphoroanilidates, ketones, sulfones, sulfonamides, carbonates, carbamates, methylenehydrazos, methylenedimethylhydrazos, formacetals, thioformacetals, oximes, methyleneiminos, methylenemethyliminos, thioamidates, linkages with riboacetyl groups, aminoethyl glycine, silyl or siloxane linkages, alkyl or cycloalkyl linkages with or without heteroatoms of, for example, 1 to 10 carbons that can be saturated or unsaturated and/or substituted and/or contain heteroatoms, linkages with morpholino structures, amides, polyamides wherein the bases can be attached to the aza nitrogens of the backbone directly or indirectly, and combinations of such modified internucleotide linkages within a polynucleotide.

Linker

A “linker” is a moiety that attaches other moieties to each other such as a nucleotide and its conjugate. A linker may be distinguished from a conjugate in that while a conjugate increases the stability and/or ability of a molecule to be taken up by a cell, a linker merely attaches a conjugate to the molecule that is to be introduced into the cell.

By way of example, linkers can comprise modified or unmodified nucleotides, nucleosides, polymers, sugars and other carbohydrates, polyethers such as, for example, polyethylene glycols, polyalcohols, polypropylenes, propylene glycols, mixtures of ethylene and propylene glycols, polyalkylamines, polyamines such as spermidine, polyesters such as poly(ethyl acrylate), polyphosphodiesters, and alkylenes. An example of a conjugate and its linker is cholesterol-TEG-phosphoramidites, wherein the cholesterol is the conjugate and the tetraethylene glycol and phosphate serve as linkers.

Nucleotide

The term “nucleotide” refers to a ribonucleotide or a deoxyribonucleotide or modified form thereof, as well as an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs.

Nucleotide analogs include nucleotides having modifications in the chemical structure of the base, sugar and/or phosphate, including, but not limited to, 5-position pyrimidine modifications, 8-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety as defined herein. Nucleotide analogs are also meant to include nucleotides with bases such as inosine, queuosine, xanthine, sugars such as 2′-methyl ribose, non-natural phosphodiester linkages such as methylphosphonates, phosphorothioates and peptides.

Modified bases refers to nucleotide bases such as, for example, adenine, guanine, cytosine, thymine, and uracil, xanthine, inosine, and queuosine that have been modified by the replacement or addition of one or more atoms or groups. Some examples of types of modifications that can comprise nucleotides that are modified with respect to the base moieties, include but are not limited to, alkylated, halogenated, thiolated, aminated, amidated, or acetylated bases, in various combinations. More specific include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles. The term nucleotide is also meant to include what are known in the art as universal bases. By way of example, universal bases include but are not limited to 3-nitropyrrole, 5-nitroindole, or nebularine.

Further, the term nucleotide also includes those species that have a detectable label, such as for example a radioactive or fluorescent moiety, or mass label attached to the nucleotide.

Nucleotide Unit

The phrase “nucleotide unit” refers to a single nucleotide residue and is comprised of a modified or unmodified nitrogenous base, a modified or unmodified sugar, and a modified or unmodified moiety that allows for linking of two nucleotides together or a conjugate that precludes further linkage.

Orthoester

The term “orthoester protected” or “orthoester modified” refers to modification of a sugar moiety in a nucleotide unit with an orthoester. Preferably, the sugar moiety is a ribosyl moiety. In general, orthoesters have the structure RC(OR′)₃ wherein R′ can be the same or different, R can be an H, and wherein the underscored C is the central carbon of the orthoester. The orthoesters of the invention are comprised of orthoesters wherein a carbon of a sugar moiety in a nucleotide unit is bonded to an oxygen, which is in turn bonded to the central carbon of the orthoester. To the central carbon of the orthoester is, in turn, bonded two oxygens, such that in total three oxygens bond to the central carbon of the orthoester. These two oxygens bonded to the central carbon (neither of which is bonded to the carbon of the sugar moiety) in turn, bond to carbon atoms that comprise two moieties that can be the same or different. For example, one of the oxygens can be bound to an ethyl moiety, and the other to an isopropyl moiety. In one example, R can be an H, one R′ can be a ribosyl moiety, and the other two R′ can be two 2-ethyl-hydroxyl moieties. Orthoesters can be placed at any position on the sugar moiety, such as, for example, on the 2′, 3′ and/or 5′ positions. Preferred orthoesters, and methods of making orthoester protected polynucleotides, are described in U.S. Pat. Nos. 5,889,136 and 6,008,400, each herein incorporated by reference in their entirety.

Overhang

The term “overhang” refers to terminal non-base pairing nucleotides resulting from one strand extending beyond the other strand within a doubled stranded polynucleotide. One or both of two polynucleotides that are capable of forming a duplex through hydrogen bonding of base pairs may have a 5′ and/or 3′ end that extends beyond the 3′ and/or 5′ end of complementarity shared by the two polynucleotides. The single-stranded region extending beyond the 3′ and/or 5′ end of the duplex is referred to as an overhang.

Pharmaceutically Acceptable Carrier

The phrase “pharmaceutically acceptable carrier” refers to compositions that facilitate the introduction of dsRNA into a cell and includes but is not limited to solvents or dispersants, coatings, anti-infective agents, isotonic agents, agents that mediate absorption time or release of the inventive polynucleotides and double stranded polynucleotides.

Polynucleotide

The term “polynucleotide” refers to polymers of nucleotides, and includes but is not limited to DNA, RNA, DNA/RNA hybrids including polynucleotide chains of regularly and irregularly alternating deoxyribosyl moieties and ribosyl moieties (i.e., wherein alternate nucleotide units have an —OH, then and —H, then an —OH, then an —H, and so on at the 2′ position of a sugar moiety), and modifications of these kinds of polynucleotides wherein the attachment of various entities or moieties to the nucleotide units at any position are included.

Polyribonucleotide

The term “polyribonucleotide” refers to a polynucleotide comprising two or more modified or unmodified ribonucleotides and/or their analogs.

Ribonucleotide and Ribonucleic Acid

The term “ribonucleotide” and the phrase “ribonucleic acid” (RNA), refer to a modified or unmodified nucleotide or polynucleotide comprising at least one ribonucleotide unit. A ribonucleotide unit comprises an oxygen attached to the 2′ position of a ribosyl moiety having a nitrogenous base attached in N-glycosidic linkage at the 1′ position of a ribosyl moiety, and a moiety that either allows for linkage to another nucleotide or precludes linkage.

RNA Interference and RNAi

The phrase “RNA interference” and the term “RNAi” refer to the process by which a polynucleotide or double stranded polynucleotide comprising at least one ribonucleotide unit exerts an effect on a biological process. The process includes but is not limited to gene silencing by degrading mRNA, interactions with tRNA, rRNA, hnRNA, cDNA and genomic DNA, as well as methylation of DNA and ancillary proteins.

Sense Strand

The phrase “sense strand” refers to a polynucleotide that has the same nucleotide sequence, in whole or in part, as a target nucleic acid such as a messenger RNA or a sequence of DNA.

siRNA or Short Interfering RNA

The term “siRNA” and the phrase “short interfering RNA” refer to a double stranded nucleic acid that is capable of performing RNAi and that is between 18 and 30 base pairs in length. Additionally, the term siRNA and the phrase “short interfering RNA” include nucleic acids that also contain moieties other than ribonucleotide moieties, including, but not limited to, modified nucleotides, modified internucleotide linkages, non-nucleotides, deoxynucleotides and analogs of the aforementioned nucleotides.

siRNAs can be duplexes, and can also comprise short hairpin RNAs, RNAs with loops as long as, for example, 4 to 23 or more nucleotides, RNAs with stem loop bulges, micro-RNAs, and short temporal RNAs. RNAs having loops or hairpin loops can include structures where the loops are connected to the stem by linkers such as flexible linkers. Flexible linkers can be comprised of a wide variety of chemical structures, as long as they are of sufficient length and materials to enable effective intramolecular hybridization of the stem elements. Typically, the length to be spanned is at least about 10-24 atoms.

Stabilized

The term “stabilized” refers to the ability of the dsRNAs to resist degradation while maintaining functionality and can be measured in terms of its half-life in the presence of, for example, biological materials such as serum. The half-life of an siRNA in, for example, serum refers to the time taken for the 50% of siRNA to be degraded.

Throughout the disclosure, where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

### PREFERRED EMBODIMENTS

According to a first embodiment, the present invention provides a double stranded polynucleotide. The double stranded polynucleotide has sense strand that comprises a polynucleotide comprised of at least one orthoester modified nucleotide, and an antisense strand that comprises a polynucleotide having at least one 2′ modified nucleotide unit. Preferably, the modified nucleotides are ribonucleotides or their analogs. Orthoesters can be placed at any position on the sugar moiety, such as, for example, on the 2′, 3′ and/or 5′ positions. Preferably, the orthoester moiety is at the 2′ position of the sugar moiety. Preferred orthoesters, and methods of making orthoester protected polynucleotides, are described in U.S. Pat. Nos. 5,889,136 and 6,008,400, each herein incorporated by reference in their entirety. Preferably, orthoesters are attached at the 2′ position of a ribosyl moiety. Preferably the orthoester comprises two 2-ethyl-hydroxyl substituents. The most preferred orthoester is illustrated below, and is also referred to herein as a 2′-ACE moiety:

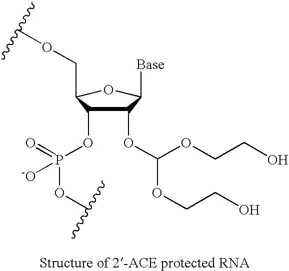

Structure of 2′-ACE Protected RNA

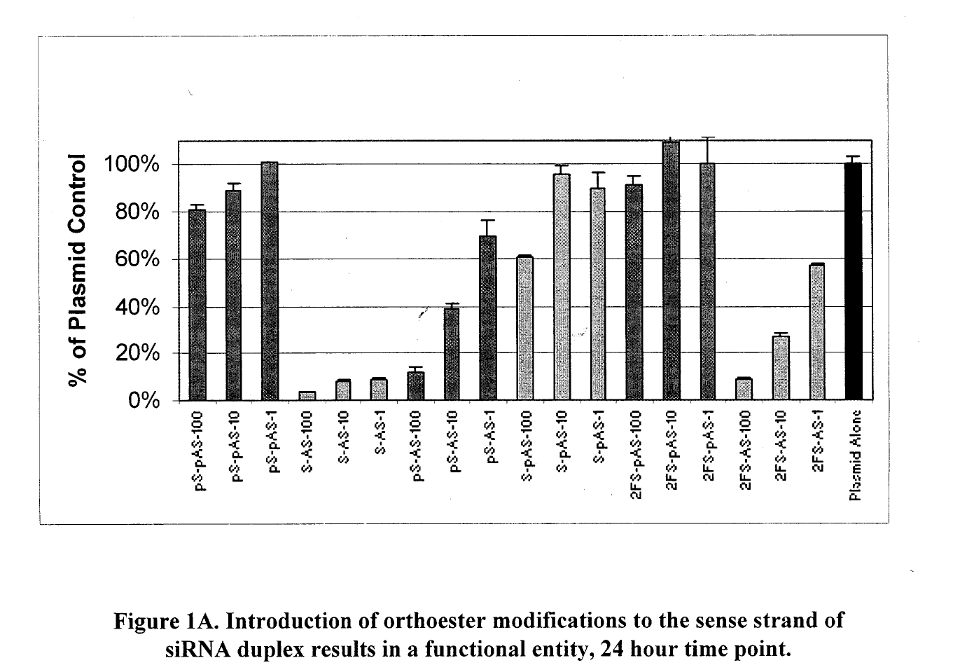

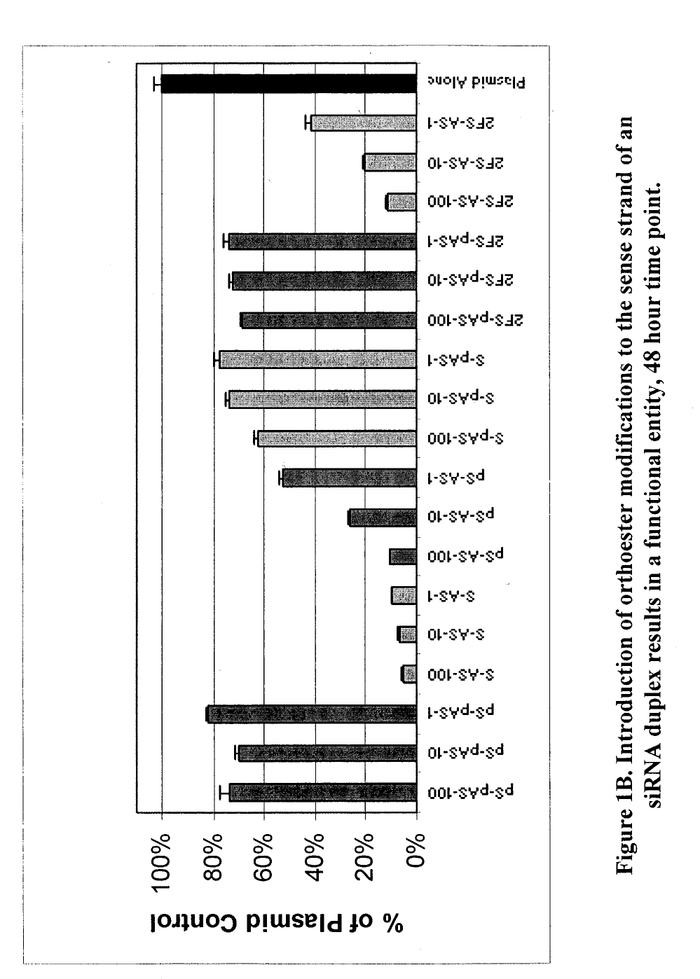

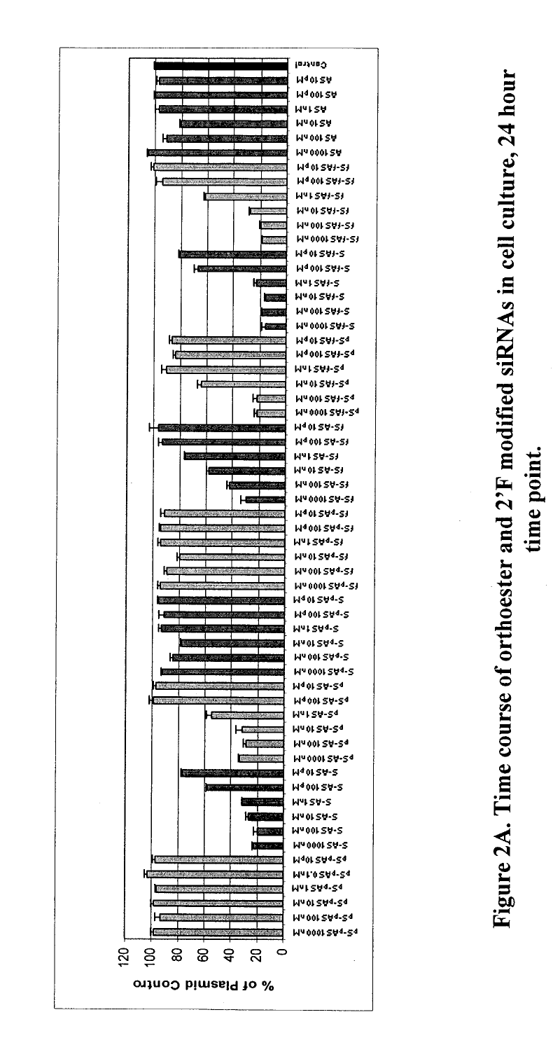

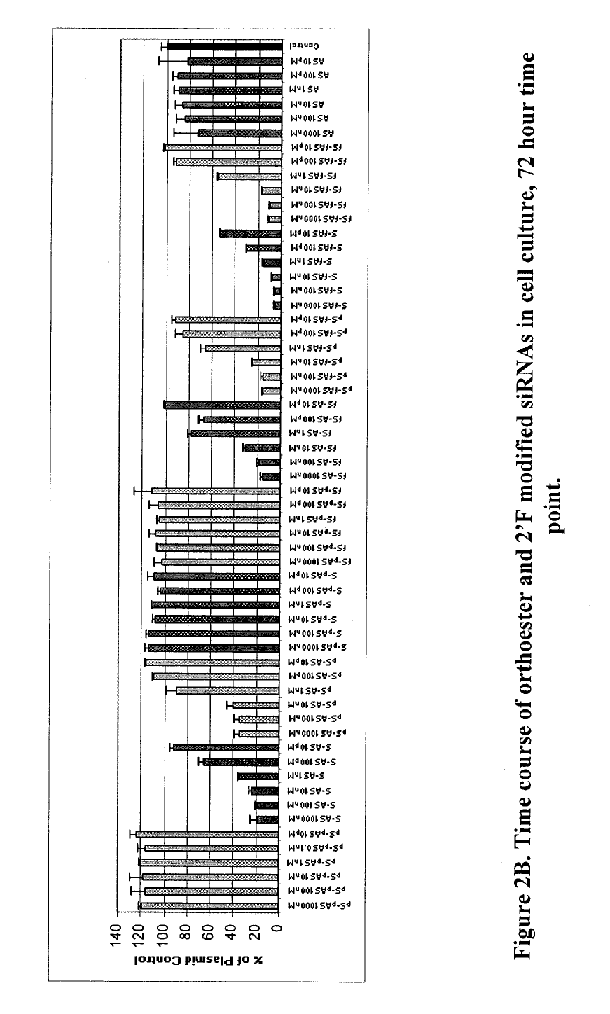

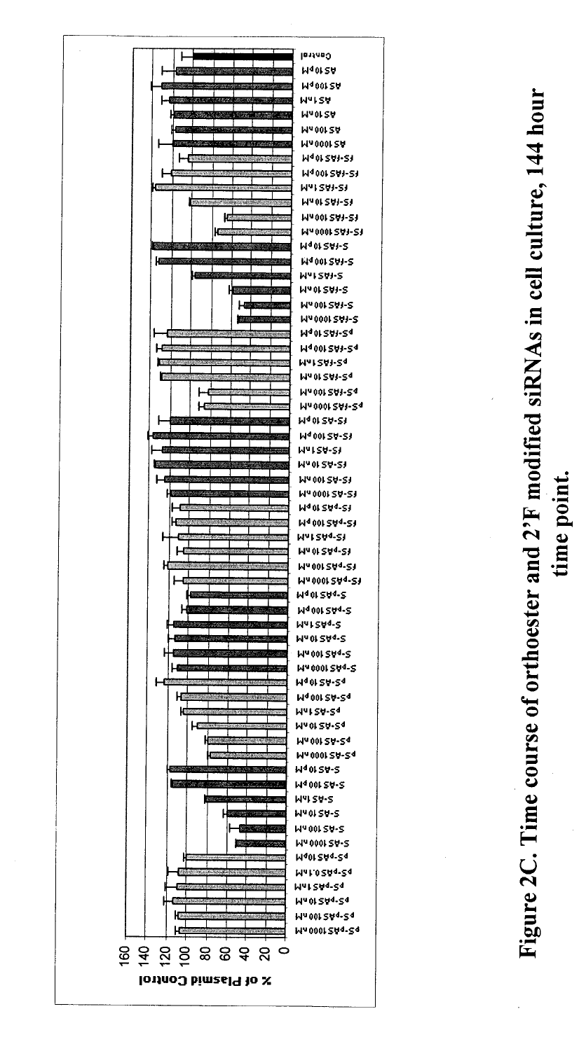

The benefits of including orthoester groups on the sense strand can be seen by reference to FIGS. 1A, 1B, 2A, 2B, and 2C.

The data of FIG. 1 were generated using an siRNA duplex targeting SEAP (human secreted alkaline phosphatase) synthesized using Dharmacon, Inc.'s proprietary ACE chemistry in several variants. These variants include naked, or unmodified, RNA; ACE protected RNA, wherein every 2′-OH is modified with an orthoester, and 2′ fluoro modified variants, wherein the fluorine is bonded to the 2′ carbon of each and every C and U.

Duplexes of siRNA can be comprised of sense and antisense strands. An array of all possible combinations of sense and antisense strands was created. With reference to the figures, the following nomenclature was used:

S—naked sense strand in an siRNA duplex

AS—naked antisense strand in an siRNA duplex

pS—2′ACE protected sense strand in an siRNA duplex

pAS—2′ACE protected antisense strand in an siRNA duplex

2FS—sense strand in an siRNA duplex with all C and U's modified such that a fluorine atom is bound to the 2′ carbon of each C- and U-bearing nucleotide unit.

2FAS—antisense strand in an siRNA duplex with all C and U's modified such that a fluorine atom is bound to the 2′ carbon or each C- and U-bearing nucleotide unit.

S—AS, refers to duplex siRNA formed from naked sense and naked antisense strands.

pS—AS, refers to duplex siRNA formed from an ACE modified sense strand and a naked antisense strand.

The duplexes were co-transfected using standard transfection protocols with the pAAV6 plasmid (SEAP expressing plasmid) (or in the HEK293s stably transfected with the SEAP) into HEK 293 human cells (the same pattern was observed when HeLas or MDA 75, or 3TELi (mouse) cell lines were used for transfection).

The level of siRNA induced SEAP silencing was determined at a different time points after transfection. (24, 48, 72, 96 or 144 hours) using SEAP detection kits from Clontech according to the manufacturer's protocols. The protein reduction levels are in good correspondence with the mRNA reduction levels (the levels of mRNA were measured using QuantiGene kits (Bayer)). The level of siRNA induced toxicity was measured using AlmaBlue toxicity assay or the levels of expression of housekeeping gene (cyclophyllin) or both. Unless specified, no significant toxicity was observed.

Each duplex was transfected into the cells at concentrations varying between 1 and 100 nanomolar (FIG. 1) and 10 picomolar to 1 micromolar (FIG. 2). In FIGS. 1 and 2 the effects of introduction of the ACE modifications on the sense and antisense strands of the siRNA duplex in combination with naked and 2′ fluoro modifications are shown.

The presence of the ACE modifications on the AS of the oligos significantly interferes with the siRNA duplex functionality. The ACE modified sense oligos were potent in the SEAP silencing independently whether they were used with naked or 2′ F modified AS oligos.

The extent of silencing was the same at 24, 48, 72 hours. The detectable reduction in the siRNA silencing was observed after 144 hours.

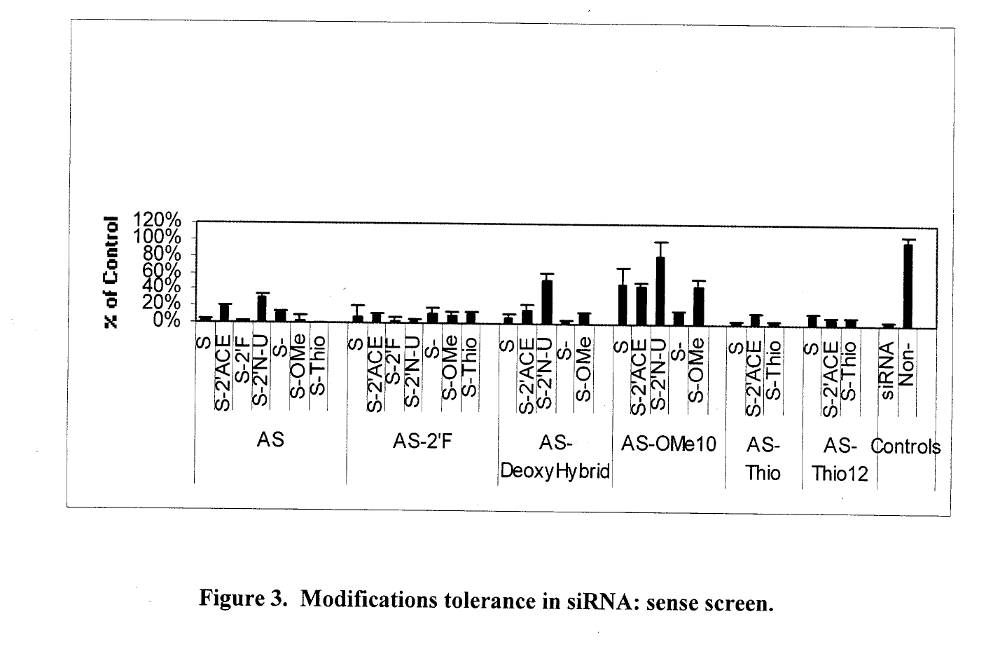

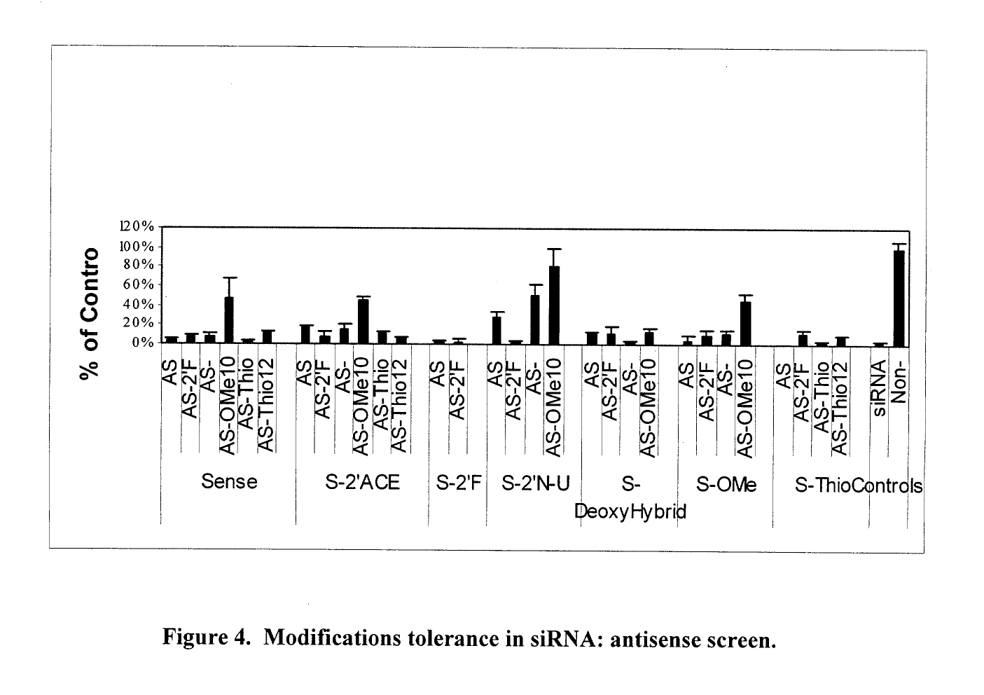

FIGS. 3 and 4 summarize siRNA functionality screens when AS (FIG. 3) or Sense (FIG. 4) strands were kept constant and screened in combination with the variety of modifications on the opposite strand.

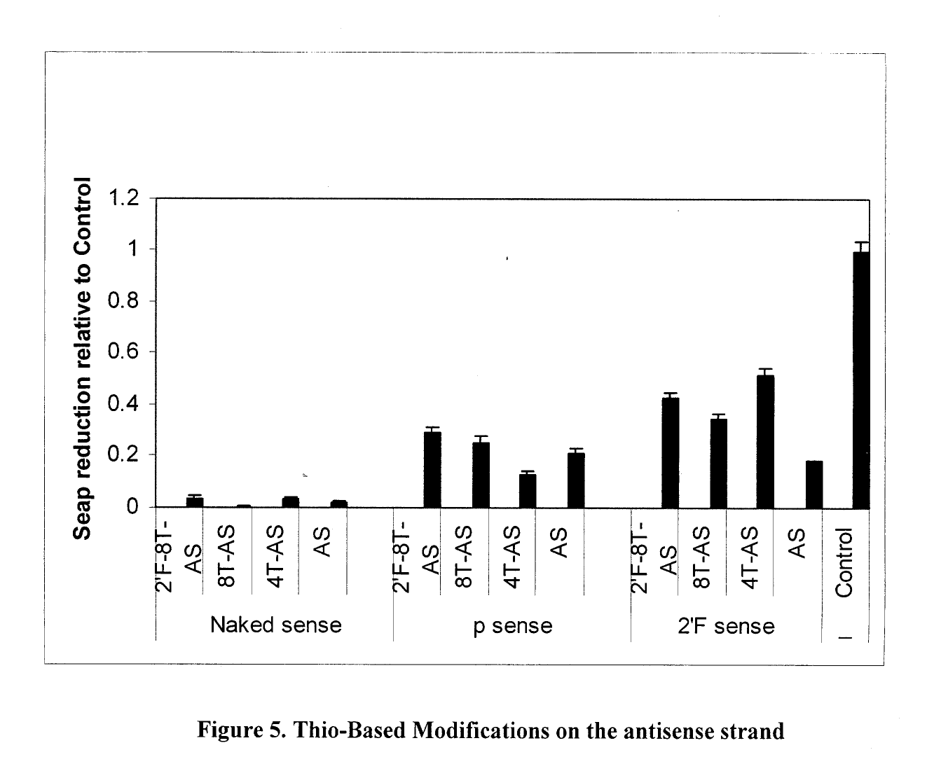

FIGS. 5, 6, 7 and 8 present a more detailed data grouped based on the type of modification used.

FIG. 5 in particular demonstrates that phosphorothioate modifications are well tolerated when placed in the antisense strand in combination with naked, 2′ACE modified and 2′F modified sense strands. The major issue with phosphorothioate modifications is well detectable toxicity observed on day 2, 3 and 4 after transfection.

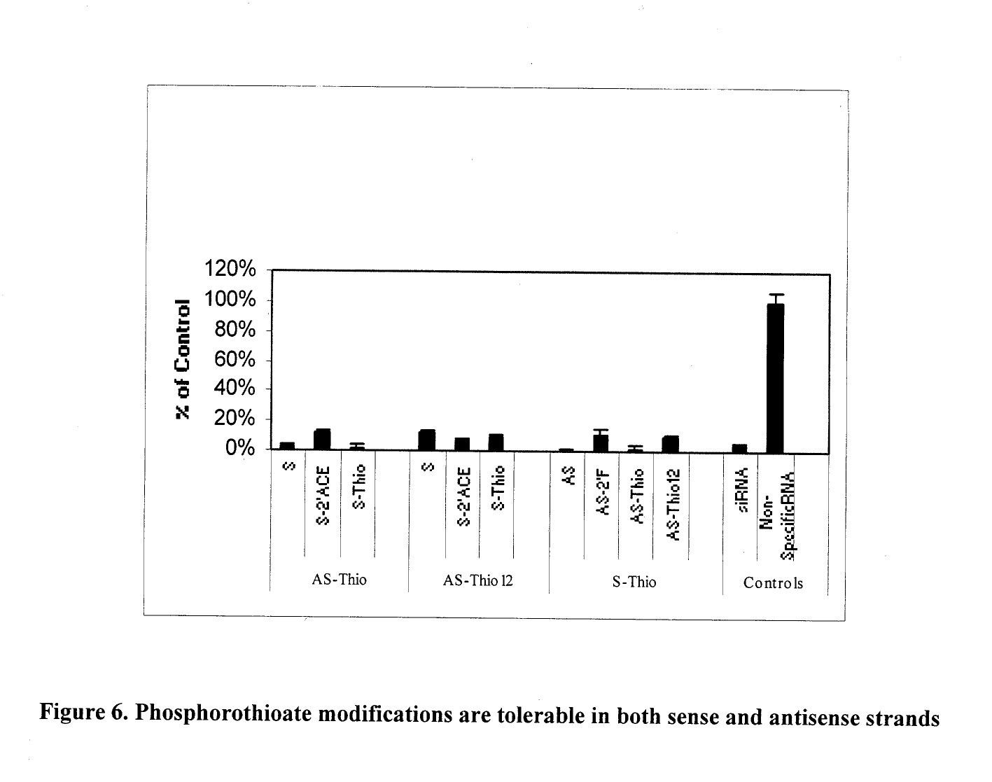

FIG. 6 further illustrates that phosphorothioate backbone modifications are acceptable both on the sense and antisense strands with the same limitation of nonspecifically induced toxicity.

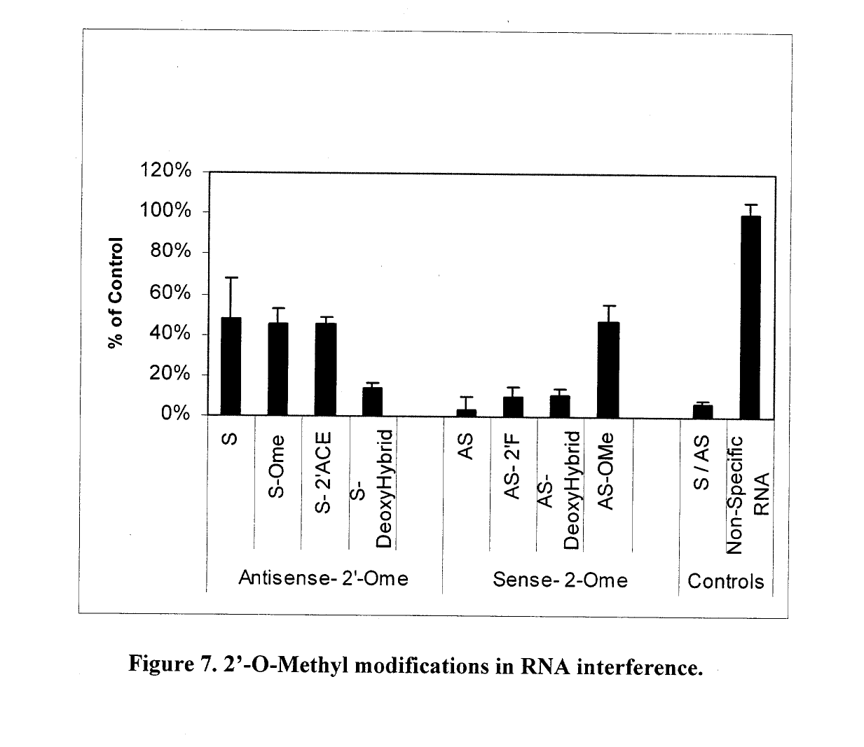

FIG. 7 demonstrated that presence of 2′-O-methyl modifications are well tolerated on sense and but not antisense strands of the siRNA duplex. It is worth mentioning that the functional siRNA duplex is formed by the combination of the 2′-O-methyl modified AS strand and deoxyribohybrid in the sense strand.

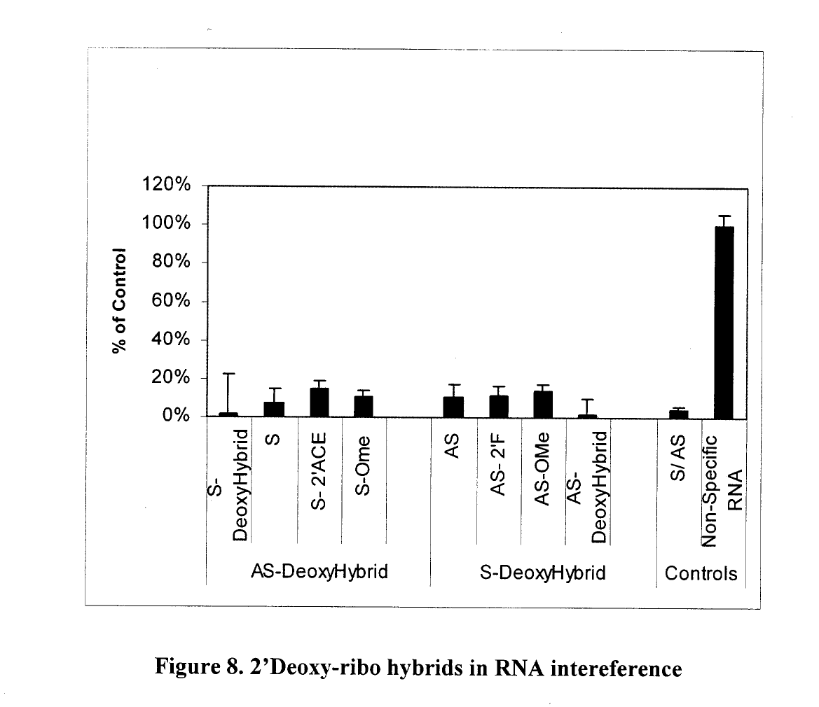

FIG. 8 demonstrates the suitability of the deoxyribohybrid type modification in RNA interference. Deoxyribohybrids are RNA/DNA hybrid oligos where deoxy and ribo entities are incorporated together in an oligo in, for example, a sequence of alternating deoxy- and ribonucleotides. It is important in the design of these kinds of oligos to keep the size of continuous DNA/RNA duplex stretches shorter than 5 nucleotides to avoid the induction of RNAse H activity. The deoxyribohybrids were functional both in sense and antisense strands in combination with 2′ fluoro and 2′ ACE modified oligos. Also the deoxyribohybrid sense strand was the only modification supporting siRNA activity when the antisense strand was modified with 2′-O-methyl.

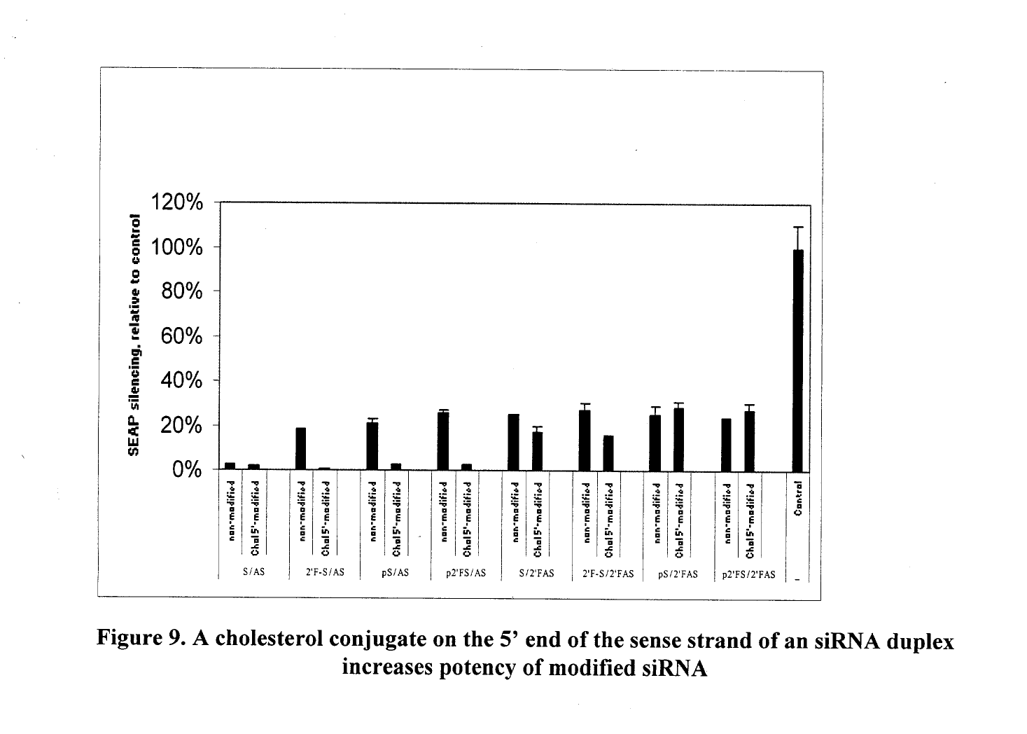

FIG. 9 demonstrates the utility of a conjugate comprising cholesterol for improvement of the potency of ACE and 2′ fluoro modified siRNAs. Employing a conjugate comprising cholesterol on the sense strand alleviates negative effects due to modifications to the sense strand, but does not ameliorate negative effects due to modifications to the antisense strand.

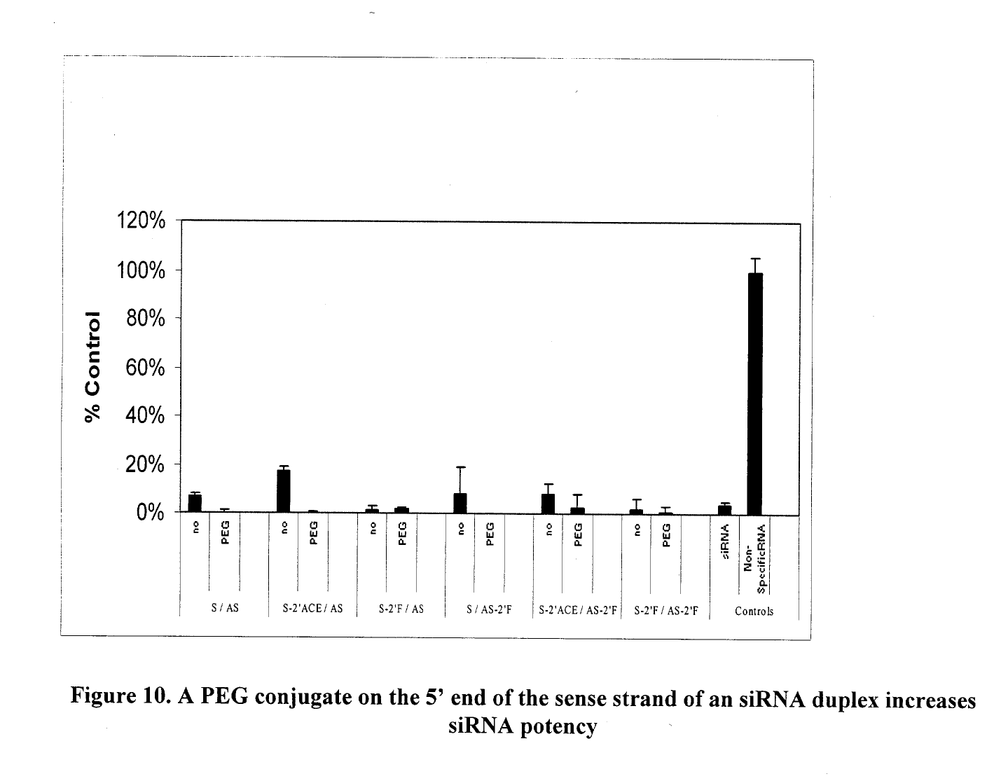

FIG. 10 shows equivalent data for a PEG conjugate on the sense strand.

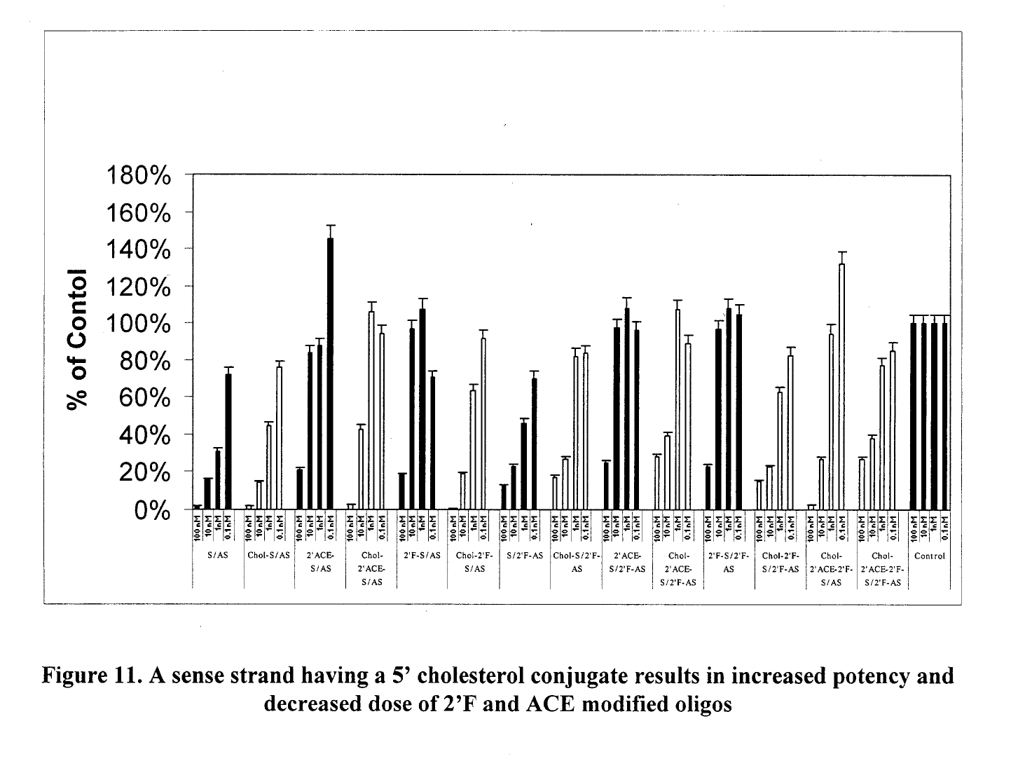

FIG. 11 demonstrates that the presence of a conjugate comprising cholesterol improves not only the potency but the effective dose of modified siRNA oligos.

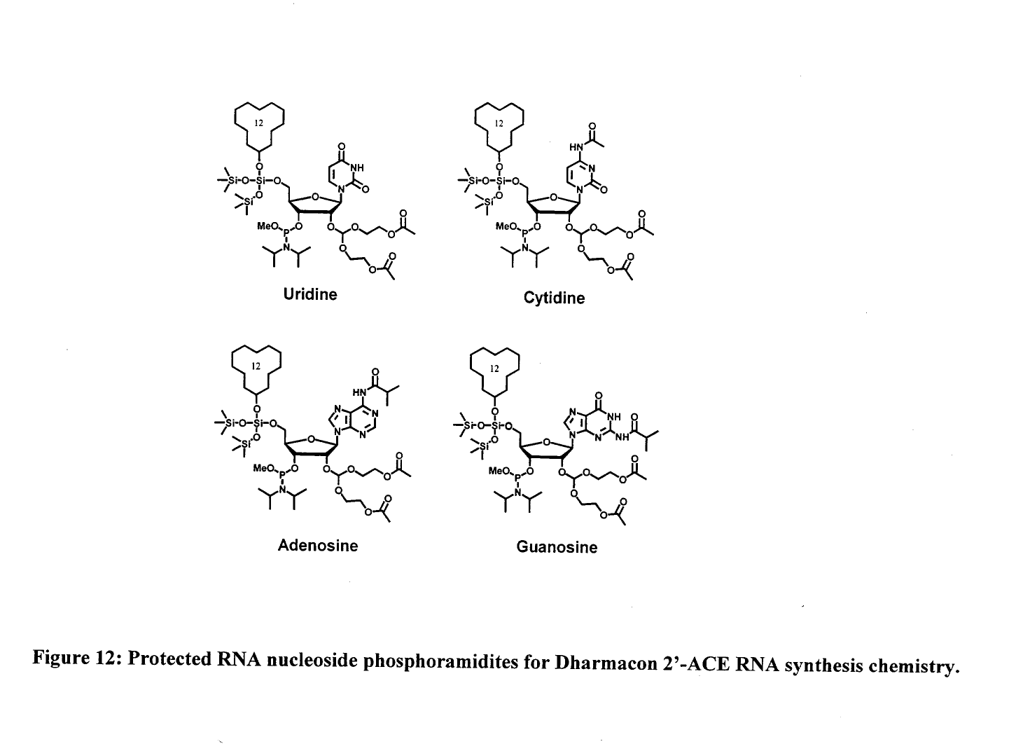

FIG. 12 shows the structures of protected RNA nucleoside phosphoramidites used in Dharmacon's 2′-ACE RNA synthesis chemistry.

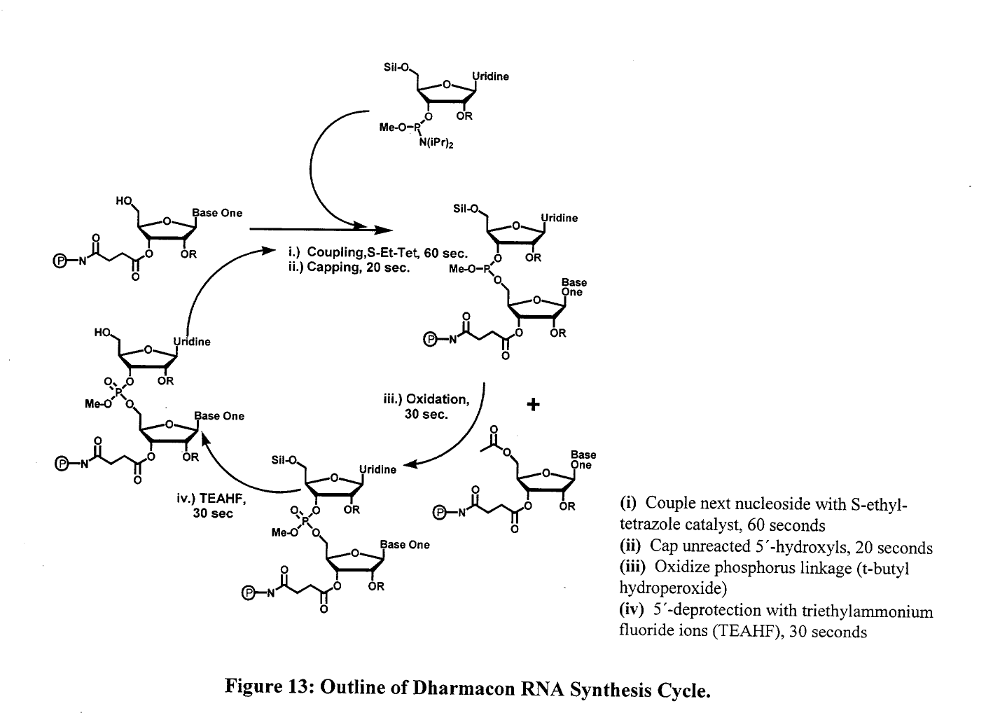

FIG. 13 outlines an RNA synthesis cycle. Preferably, the cycle is carried out in an automated fashion on a suitable synthesizing machine. In step (i), the incoming phosphoramidite (here, bearing a uridine as nitrogenous base), can bear any acceptable group on the phosphoramidite moiety at the 3′ position in place of the methyl group shown. For example, an alkyl group or a cyanoethyl group can be employed at that position. This RNA synthesis cycle can be carried out, with certain changes, when synthesizing polynucleotides having modified internucleotide linkages, and/or when synthesizing polynucleotides having other modifications, such as at the 2′ position, as described hereinafter.

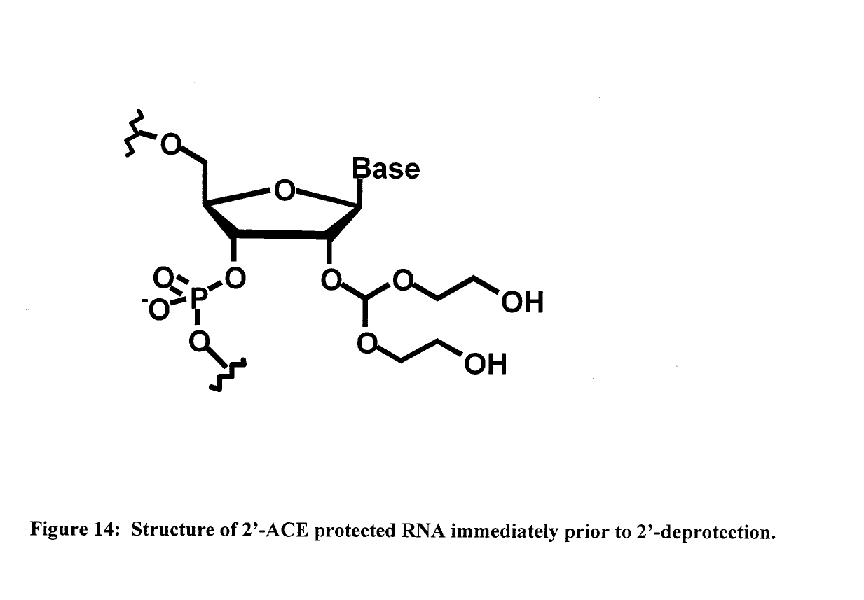

FIG. 14 illustrates the structure of a 2′-ACE protected RNA product immediately prior to 2′ deprotection. If it is desired to retain the orthoester at the 2′ position, this 2′ deprotection step is not carried out.

For a 19-mer duplex having a di-ddT overhang at both the 5′ and 3′ end, AC 2′-n-U c 2′-n-u C u g a c a 2′-N—U A c a 2′-N—U c a c dT dT (SEQ. ID NO. 64) with 2′ amine modified nucleotide units at the second, fourth, twelfth, and sixteenth position of the sense strand, significant loss in functionality occurred whether the antisense strand was naked, 2′ fluoro modified at all C's and U's, was a deoxyhybrid comprising alternating ribo and deoxyribonucleotide units, or had 2′-O-methyl modifications. Preferably, the sense strand does not comprise 2′ amino modifications at the second, fourth, twelfth and sixteenth positions.

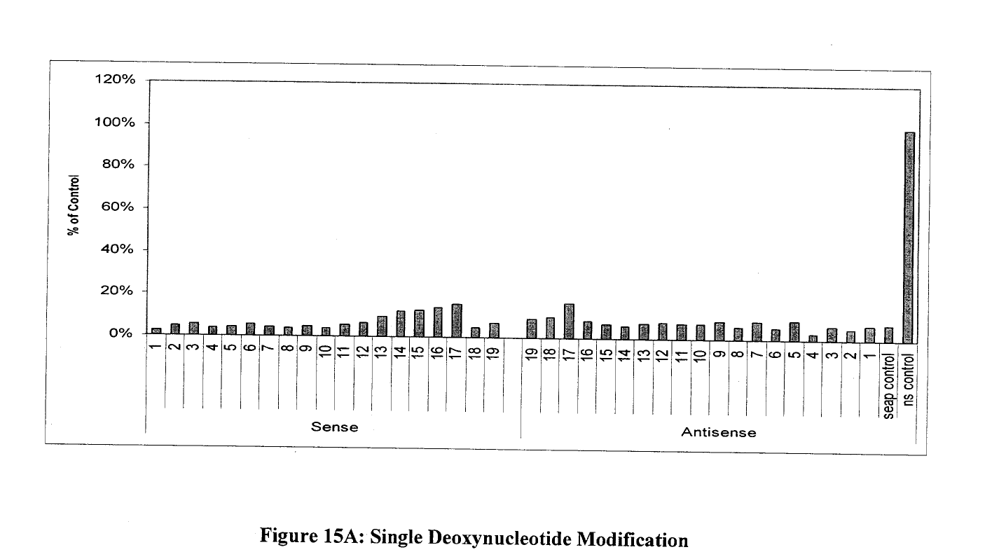

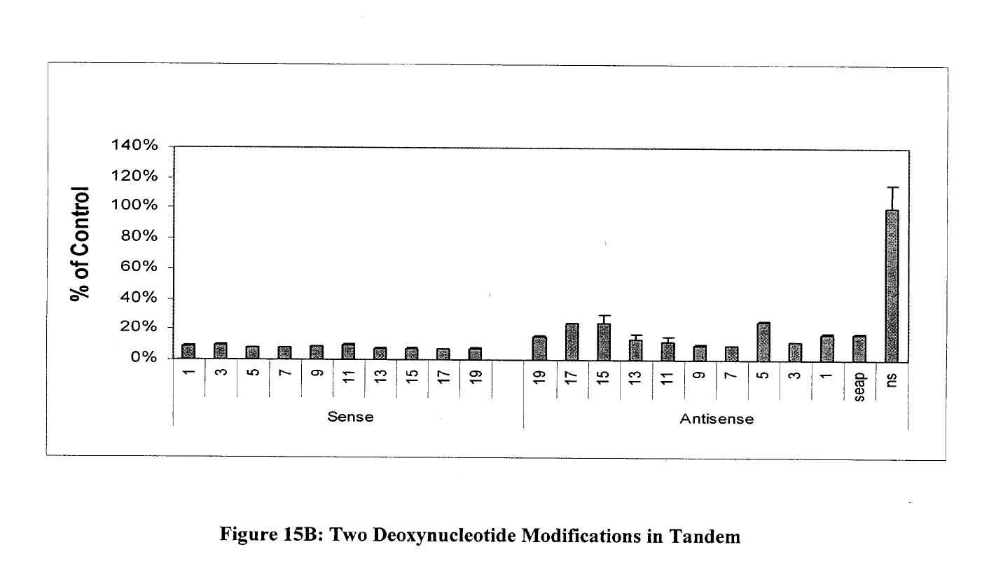

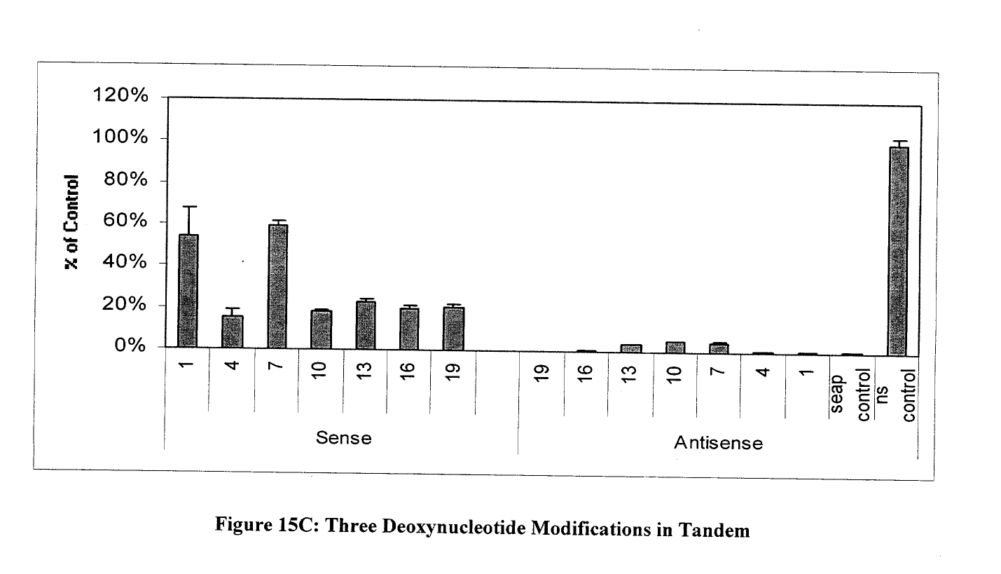

On a double stranded 19-mer polyribonucleotide with a 3′ di-dT overhang (see SEQ. ID NOs. 26-63), replacement of any ribonucleotide unit with a deoxyribonucleotide unit does not significantly affect the functionality of the 19-mer in RNAi, whether the modification is on the sense or the antisense strand (see FIG. 15A). On the same double stranded 19-mer, replacement of two adjacent ribonucleotide units with two deoxyribonucleotide units in tandem does not significantly affect the functionality of the 19-mer in RNAi. FIG. 15B illustrates that when positions 1 and 2, 3 and 4, 5 and 6, and so on, are independently modified to be deoxyribonucleotides, functionality is not significantly affected when the modifications are borne on the sense strand and exhibit only a slight negative effect on functionality when the modifications are on the antisense strand. On the same double stranded 19-mer, replacement of three adjacent ribonucleotide units with three deoxyribonucleotide units in tandem does not significantly affect the functionality if the modification is on the antisense strand, but can significantly affect functionality if the modified units are the first through third or seventh through ninth units. In this experiment, units 1 to 3, 4 to 6, 7 to 9, and so on of the polyribonucleotide were independently replaced with deoxyribonucleotide units (See FIG. 15C).

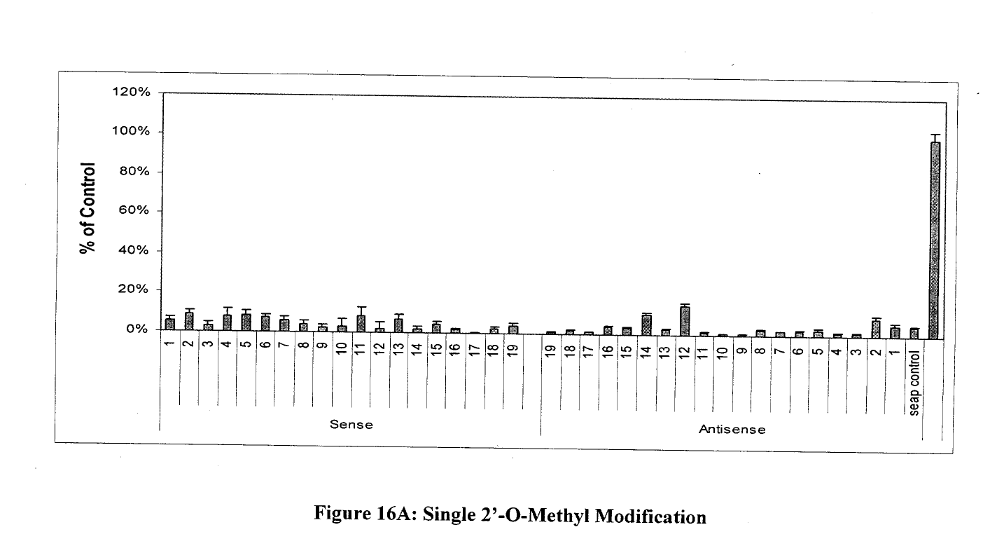

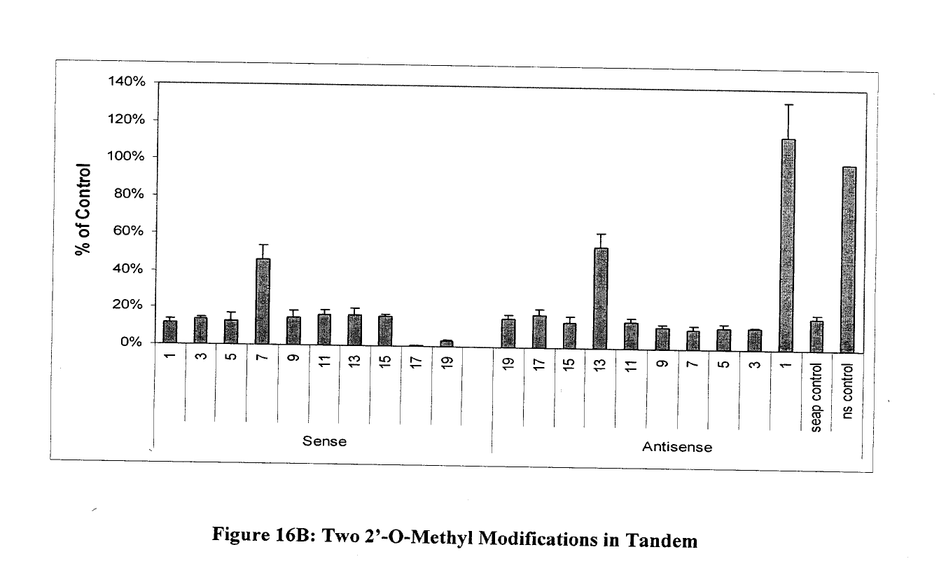

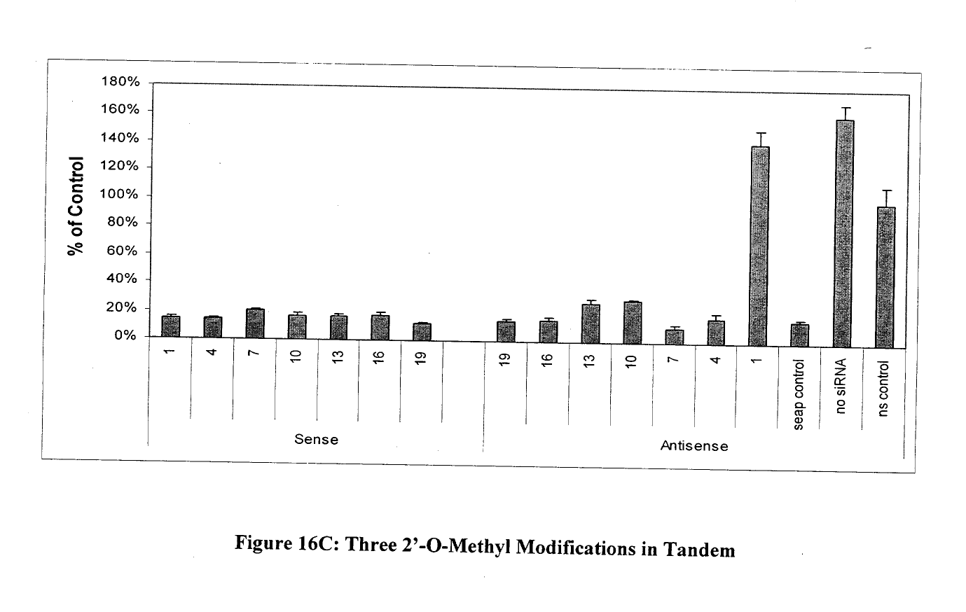

On the same double stranded 19-mer polyribonucleotide with 3′ di-dT overhang, modification of any individual unit with a 2′-O-methyl moiety does not significantly affect the functionality of the 19-mer in RNAi, whether the modification is on the sense or the antisense strand (see FIG. 16A). Using the same the same double stranded 19-mer, replacement of two adjacent ribonucleotide units with two 2′-O-methyl modifications in tandem does not significantly affect the functionality of the 19-mer in RNAi unless the modifications are placed at the first and second or thirteenth and fourteenth positions of the antisense strand, or the seventh and eighth position of the sense strand (see FIG. 16B). Most notably, the first and second positions of the antisense strand should not bear 2′-O-methyl modifications if functionality is to be preserved. Using the same double stranded 19-mer, replacement of three adjacent ribonucleotide units with 2′-O-methyl modifications in tandem does not significantly affect the functionality if the modifications are on the antisense strand at positions other than the first through third positions (See FIG. 16C). In this experiment, positions 1 to 3, 4 to 6, 7 to 9, and so on of the polyribonucleotide were independently modified with 2O-methyl moieties.

Modification of the same polyribonucleotide with either a single 2′-deoxy moiety or a single 2O-methyl moiety has no significant affect on functionality. Modification of the first and second or first, second and third positions of the antisense strand with two or more tandem 2′-O-methyl moieties can significantly reduce functionality. Positions 7 through 9 on the sense strand and 13 through 15 on the antisense strand are sensitive to two or more tandem 2′-O-methyl modifications. Thus, preferably the antisense strand does not comprise 2′-O-methyl modifications at the first and second; the first, second and third; the thirteenth and fourteenth; and the thirteenth, fourteenth and fifteenth positions.

As a matter of practicality it is more economical to synthesize a sense strand in which all of the nucleotides are modified by an orthoester group, rather than a sense strand in which only selected nucleotides are so modified. However, in theory, if a practical means were developed to synthesize sense strands in which only certain nucleotides were modified, then those polynucleotides could be used in the present invention.

Preferably, the 2′ modified nucleotide is selected from the group consisting of a 2′ halogen modified nucleotide, a 2′ amine modified nucleotide, a 2′-O-alkyl modified nucleotide, and a 2′ alkyl modified nucleotide. Where the modification is a halogen, the halogen is preferably fluorine. When the modification is fluorine, preferably it is attached to one or more nucleotides comprising a cytosine or a uracil base moiety. Where the 2′ modified nucleotide is a 2′ amine modified nucleotide, the amine is preferably —NH₂. Where the 2′ modified nucleotide is a 2′-O-alkyl modification, preferably the modification is a 2′-O-methyl, ethyl, propyl, isopropyl, butyl, or isobutyl moiety and most preferably, the 2′-O-alkyl modification is a 2′-O-methyl moiety. Where the 2′ modified nucleotide is a 2′-alkyl modification, preferably the modification is a 2′ methyl modification, wherein the carbon of the methyl moiety is attached directly to the 2′ carbon of the sugar moiety.

For modifications of the 2′ group on the antisense strand, preferably no modification will appear at positions 8-11, and more preferably positions 7-12 will be unmodified. The positions are preferably not modified because they must retain the ability to recognize the protein complex associated with RNAi.

FIG. 2C demonstrates that siRNA effects start to fade out 144 hours after transfection. The dose as well as potency of the modified oligos were comparable to the naked siRNA duplex.

According to a second embodiment, the present invention provides a double stranded polynucleotide comprising a sense strand where the sense strand comprises a polynucleotide having at least one orthoester modified nucleotide as provided for according to the first embodiment; an antisense strand comprising a polynucleotide that has at least one 2′ modified nucleotide as provided for according to the first embodiment; and a conjugate.

The conjugate within this embodiment is preferably selected from the group consisting of amino acids, peptides, polypeptides, proteins, sugars, carbohydrates, lipids, polymers, nucleotides, polynucleotides, and combinations thereof. More preferably it is selected from the group consisting of cholesterol, polyethylene glycol, antigens, antibodies, and receptor ligands. Even more preferably, the conjugate comprises cholesterol or polyethylene glycol. Most preferably, the conjugate comprises cholesterol and is linked to the 5′ terminal nucleotide unit of the sense strand at the 5′ position.

Introduction of a cholesterol-containing conjugate at the 5′ terminus of the sense strand resulted in an increase in potency for orthoester modified and 2′ antisense modified siRNAs that was comparable to or even superior to the naked, or unmodified, duplexes. See FIG. 9 and 11. A 5′ cholesterol modification of the sense strand resulted in a decrease in the functionally effective dose for orthoester modified and 2′ fluorine modified siRNAs that were comparable or even superior to the corresponding naked duplexes.

FIG. 9 demonstrates the utility of the cholesterol modification for improvement of the potency of ACE and 2′ fluoro modified siRNAs. The positive cholesterol effect was observed with the modifications introduced mainly on the sense and non antisense strands.

FIG. 10 shows equivalent data for PEG sense strand modifications.

FIG. 11 demonstrates that the presence of cholesterol modifications improves not only the potency but the effective dose of modified siRNA oligos Preferably, a single conjugate is employed. Most preferably, the conjugate is attached to the 5′ terminus of the sense strand. In order of decreasing preference, the single conjugate can be attached to the 3′ terminus of the sense strand, the 3′ terminus of the antisense strand, and the 5′ terminus of the antisense strand.

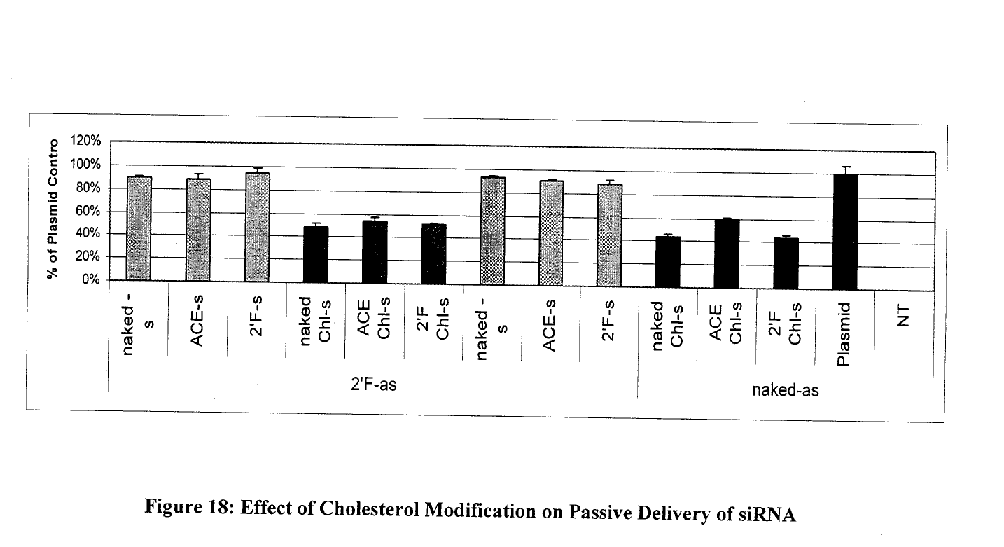

Attachment of a conjugate to an siRNA can promote uptake of the siRNA passively, that is, in the absence of transfection agents such as lipids or calcium chloride. For example, attachment of a cholesterol moiety to the 5′ end at the 5′ position of the sense strand of SEQ. ID NOs. 1-16 results in RNAi in the absence of transfection agents (see FIG. 18).

According to a third embodiment, the present invention provides a double stranded polynucleotide that has a sense strand comprised of at least one orthoester modified nucleotide, an antisense strand, and a conjugate. In this embodiment, the orthoester modification of the first embodiment may be used in combination with the conjugate of the second embodiment.

According to a fourth embodiment, the present invention provides a double stranded polynucleotide that has a sense strand, an antisense strand, and a conjugate, wherein the sense strand and/or the antisense strand has at least one 2′ modified nucleotide. The 2′modified nucleotide of this embodiment is preferably selected according to the same parameters as the 2′modified nucleotide of the first embodiment. Similarly, the conjugate is preferably selected according to the same parameters as the conjugate is selected in the above described second embodiment.

According to a fifth embodiment, the present invention provides a double stranded polyribonucleotide having a sense strand comprised of at least one orthoester modified nucleotide, an antisense strand comprised of at least one 2′ modified nucleotide selected from the group consisting of a 2′ halogen modified nucleotide, a 2′ amine modified nucleotide, a 2′-O-alkyl modified nucleotide, and a 2′ alkyl modified nucleotide, and a conjugate selected from the group consisting of amino acids, peptides, polypeptides, proteins, sugars, carbohydrates, lipids, polymers, nucleotides, polynucleotides, and combinations thereof, wherein the polyribonucleotide comprises between 18 and 30 nucleotide base pairs.

The orthoester of this embodiment is selected according to the criteria for selecting the orthoester of the first embodiment. Where the 2′ modification is a halogen, preferably it is fluorine and is attached to at least one C- and U-containing nucleotide units of the antisense strand. Where the 2′ modified nucleotide is a 2′ amine modified nucleotide, the amine is preferably —NH₂. Where the 2′ modified nucleotide is a 2′-O-alkyl modification, preferably it is a 2′-O-methyl, ethyl, propyl, isopropyl, butyl, or isobutyl moiety and most preferably, the 2′-O-alkyl modification is a 2′-O-methyl moiety. Where the 2′ modified nucleotide is a 2′ alkyl modification, preferably it is a 2′ methyl modification, wherein the carbon of the methyl moiety is attached directly to the 2′ carbon of the sugar moiety.

According to a sixth embodiment, the present invention includes a composition comprising the structures below:

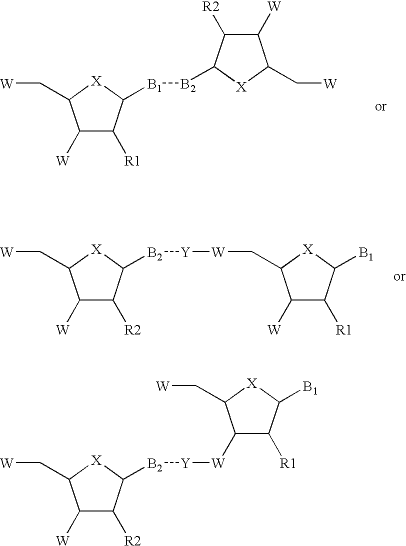

wherein each of B₁ and B₂ is a nitrogenous base, heterocycle or carbocycle; X is selected from the group consisting of O, S, C, and N; W is selected from the group consisting of an OH, a phosphate, a phosphate ester, a phosphodiester, a phosphotriester, a modified internucleotide linkage, a conjugate, a nucleotide, and a polynucleotide; R1 is an orthoester; R2 is selected from the group consisting of a 2′-O-alkyl group, an alkyl group, an amine, and a halogen; and Y is a nucleotide or polynucleotide. Where R2 is a halogen, the halogen is preferably a fluorine. Where R2 is a fluorine, the fluorine is preferably attached to one or more C- and U-containing nucleotide units. Where R2 is an amine, the amine is preferably —NH₂. Where R2 is a 2′-O-alkyl modification, preferably it is a 2′-O-methyl, ethyl, propyl, isopropyl, butyl, or isobutyl moiety and most preferably a 2′-O-methyl moiety. Where R2 is a 2′ alkyl modification, preferably it is a 2′ methyl modification, wherein the carbon of the methyl moiety is attached directly to the 2′ carbon of the sugar moiety.

R1, the orthoester, of this embodiment is selected according to the parameters for selecting the orthoester of the first embodiment.

The dashed lines in the formula indicate interaction by hydrogen bonding between nitrogenous bases. Preferably, B₁ and B₂ are naturally occurring nitrogenous bases such as, for example, adenine, thymine, guanine, cytosine, uracil, xanthine, hypoxanthine, and queuosine or analogs thereof. Preferably, X is an O.

With respect to each of the above-described embodiments, the double stranded polynucleotides can be of any length, but preferably are 18-30 nucleotide base pairs, more preferably 18-19 base pairs, excluding any overhang. By using double stranded polynucleotides of less than about 30 base pairs in length one can avoid nonspecific processes, such as interferon-related responses, which can reduce the functionality of an siRNA application, while retaining a functional response in RNA interference applications. Additionally, preferably the nucleotides are ribonucleotides.

In the above-described embodiments, overhangs can be present on either or both strands, at either or both ends. Preferably, if a double stranded polynucleotide has overhang, it is one to six nucleotide units in length, more preferably two to three, and most preferably two, and is located at the 3′ end of each strand of the double stranded polynucleotide. However, siRNAs with blunt ends are functional. Overhangs of 2 nucleotides are most preferred.

Similarly in the above-described embodiments, either or both strands of the double stranded polynucleotide can have one or more modified internucleotide linkages. Preferably, the modified internucleotide linkages are selected from the group consisting of phosphorothioates and phosphorodithioates. Additionally, preferably, the polynucleotides comprise more than 4 modified internucleotide linkages. More preferably, the polynucleotides of the invention comprise more than 8 modified internucleotide linkages. Most preferably, about 10 modified internucleotide linkages are employed. For the greatest amount of stability, complete modification is preferred; however, a number of factors affect how many modified linkages can be employed in practice. These factors include the degree of stability conferred by the linkage, the degree to which the linkage affects functionality, the ability to introduce the linkage chemically, and the toxicity of the linkage. Preferably, modifications are localized on the 3′ and 5′ ends to protect against exonuclease activity.

The polynucleotides of the present invention are stabilized. The half-lives of the stabilized siRNA of the invention are from 20 seconds to 100 or more hours. Preferably, the stabilized siRNAs of the invention display half-lives of 1 to 10 hours. More preferably, the stabilized siRNAs of the invention display half-lives of 11 to 100 hours. Most preferably, the stabilized siRNAs of the invention display half-lives in excess of 100 hours. Additionally, preferably the effect of the siRNAs will survive cell division for at least one or more generations.

The polynucleotides of the invention exhibit enhanced stability in the presence of human serum. Preferably, the half life of a 19-mer duplex in human serum is from several minutes to 24 hours. More preferably, the half life of a 19-mer duplex in human serum is from 24 hours to 3 days. Most preferably, the half life of a 19-mer duplex in human serum if from 3 to 20 or more days.

For a 19-mer polyribonucleotide duplex comprising an antisense strand with deoxyribonucleic modifications at the second, fourth, sixth, fourteenth, sixteenth, and eighteenth positions, exposure to fetal bovine serum for half an hour at 37 degrees Centigrade resulted in protection of the fourth and sixth positions from degradation, presumably by serum nucleases. Similarly, for a 19-mer polyribonucleotide duplex comprising 2′-O-methyl modifications on the antisense strand at the second through sixth, twelfth, fourteenth, sixteenth and seventeenth, and nineteenth positions resulted in protection of these positions from degradation by serum nucleases. Introduction of phosphorothioate modifications in the antisense strand for a 19-mer polyribonucleotide duplex at between nucleotide units one through six and thirteen through nineteen rendered the modified internucleotide linkages resistant to serum nuclease degradation. However, a 19-mer modified with an ACE orthoester moiety at each 2′ position of an antisense strand did not confer stability in human serum, presumably due to the action not of serum ribonucleases but of serum phosphodiesterases.

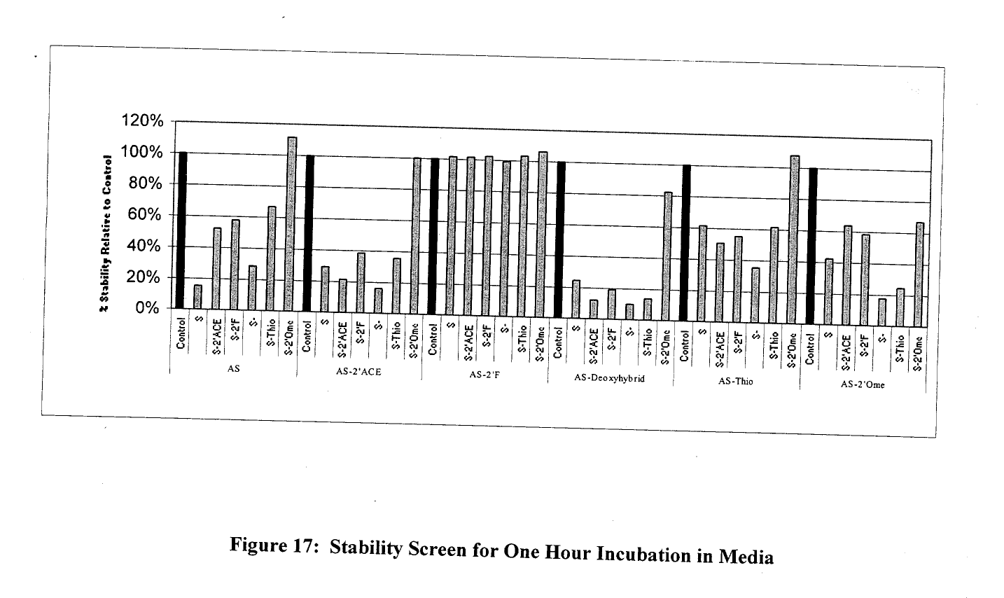

Modifications at the 2′ position in the antisense strand of a polyribonucleotide duplex, at C and U nucleotide units, greatly enhance the stability of the polyribonucleotide duplex in serum. FIG. 17 illustrates stability as a function of type of modification at the 2′ position on both the sense and antisense strands for 2′-O-methyl (SEQ. ID NO. 13), for2′ F(5′-2° F_G fU G A fU G fU A fU G fU fC A G A G A G fU dT dT-3′) (SEQ. ID NO. 65); for phosphorothioate internucleotide linkages (SEQ. ID. NOs. 10 and 11) and for ACE-protected (SEQ. ID. NOs. 3 and 4). The vertical axis represents the percent of nondegraded polynucleotide versus a control. Thus, the higher the percent stability relative to control, the less degradation observed. From FIG. 17 it is apparent that modifying the sense strand is sufficient to achieve stabilization.

Modification of each C and each U with either a 2′-O-methyl moiety or a 2′ fluoro moiety results in complete stabilization of the sense and the antisense strand. Annealing a stable sense strand, such as one having 2′ fluoro or 2′-O-methyl modifications, to a naked antisense strand results in improved stability.

The compositions of the invention can be made according to Dharmacon's RNA synthesis chemistry, which is based on a novel protecting group scheme. A new class of silyl ethers is used to protect the 5′-hydroxyl (5′-SIL) in combination with an acid-labile orthoester protecting group on the 2′-hydroxyl (2′-ACE). This set of protecting groups is then used with standard phosphoramidite solid-phase synthesis technology. The structures of some protected and functionalized ribonucleotide phosphoramidites are as illustrated in FIG. 12.

According to a seventh embodiment, the present invention provides a method of performing RNA interference. This method is comprised of exposing a double stranded polynucleotide to a target nucleic acid in order to perform RNAi. Under this method, the double stranded polynucleotide is comprised of a sense strand and an antisense strand, and at least one of said sense strand and said antisense strand comprises at least one orthoester modified nucleotide.

Preferably, the polynucleotides of the antisense strand exhibit 90% or more complementarity to the target nucleic acid of interest. More preferably, the polynucleotides antisense strand of the invention exhibit 99% or more complementarity to the target nucleic acid of interest. Most preferably, the polynucleotides of the invention are perfectly complementary to the target nucleic acid of interest over at least 18 to 19 contiguous bases.

Preferably, the at least one orthoester modified nucleotide is located on the sense strand, and the composition of the orthoester is defined by the parameters described above for the first embodiment.

In addition to the orthoester modification, any of the above described other modifications may also be present when using this method. For example, the antisense strand preferably comprises at least one modified nucleotide selected from the group consisting of a 2′ halogen modified nucleotide, a 2′ amine modified nucleotide, a 2′-O-alkyl modified nucleotide and a 2′ alkyl modified nucleotide. Where the modified nucleotide is a 2′ halogen modified nucleotide, the halogen is preferably a fluorine. Where the halogen is a fluorine, the fluorine is preferably attached to C- and U-containing nucleotide units. Where the 2′ modification is an amine, preferably the amine is —NH₂. Where the 2′ modification is a 2′-O-alkyl group, preferably the group is methoxy, —OCH₃. Where the 2′ modification is an alkyl group, preferably the modification is a methyl group, —CH₃. Further, preferably none of these modifications occur at nucleotides 8-11, and more preferably none of the occur at positions 7-12 of the antisense strand.

The method can also be carried out wherein the double stranded polynucleotide comprises a 5′ conjugate. The conjugate can be selected according to the above-described criteria for selecting conjugates.

When using these methods, the double stranded polynucleotide can be of any number of base pairs, but is preferably is 18-30 base pairs, and more preferably is 19 base pairs. Additionally preferably the polynucleotide comprises an antisense strand and a sense strand of ribonucleotides.

Overhangs of one or more base pairs at the 3′ and/or 5′ terminal nucleotide units on either or both strands can also be present according to the above-described parameters for overhangs.

According to an eighth embodiment, the present invention provides a method of performing RNA interference, comprised of exposing a double stranded polynucleotide to a target nucleic acid, wherein the double stranded polynucleotide is comprised of a sense strand, an antisense strand, and a conjugate, where either the sense strand or the antisense strand comprises a 2′ modified nucleotide. Preferably, the polynucleotides of this embodiment of the invention exhibit the same degree of complementarity as in the previous example.

According to this embodiment, the antisense strand preferably comprises at least one nucleotide selected from the group consisting of a 2′ halogen modified nucleotide, a 2′ amine modified nucleotide, a 2′-O-alkyl modified nucleotide and a 2′ alkyl modified nucleotide. The modification may be on the antisense strand and/or on the sense strand. Where the modified nucleotide is a 2′ halogen modified nucleotide, the halogen is preferably fluorine. Where the halogen is fluorine, the fluorine is preferably attached to at least one C- or U-containing nucleotides. The preferred 2′ amine modification is —NH₂. The preferred 2′-O-alkyl modification is —OCH₃. The preferred 2′ alkyl modification is —CH₃.

The method can also be carried out wherein the double stranded polynucleotide comprises a conjugate. The conjugate is selected according to the parameters for selecting the above-described conjugates. The double stranded polynucleotide can be of any number of base pairs, but as with the previous embodiment is preferably 18-30 base pairs, most preferably 18-19 base pairs. Similarly, overhangs of one or more base pairs on the 3′ and/or 5′ terminal nucleotide units on either or both strands can be present. Further, either the sense or antisense strand can comprise at least one modified internucleotide linkage, which preferably is selected from the group consisting of phosphorothioate linkages and phosphorodithioate linkages. Preferably the sense and antisense strands are polyribonucleotides.

Each of the aforementioned embodiments permits the conducting of efficient RNAi interference because the polynucleotide is more stable than naked polynucleotides. Unlike naked polynucleotides, the polynucleotides of the present invention will resist degradation by nucleases and other substances that are present in blood, serum and other biological media.

An additional surprising benefit of the present invention is that it minimizes nonspecific RNA interference. Nonspecific RNA interference occurs when a sense strand silences or partially silences the function of untargeted genes. Orthoester modifications and the other modifications described herein, alone or in combination with one another, can be employed in the sense strand to reduce or prevent such nonspecific RNA interference.

In reducing nonspecific RNA interference, preferably sense strand modifications are made at the 2′ position at the 8^{th}, 9^{th}, 10^{th}, or 11^{th} nucleotide from the 5′ terminus, with the 5′ terminal nucleotide designated as the 1^{st}. More preferably, all of the 8^{th}, 9^{th}, 10^{th} and 11^{th} nucleotides are modified at the 2′ position. Most preferably, the 8^{th}, 9^{th}, 10^{th} and 11^{th} nucleotides are all modified at the 2′ position and the modification is an orthoester.

Once synthesized, the polynucleotides of the present invention may immediately used or be stored for future use. Preferably, the polynucleotides of the invention are stored as duplexes in a suitable buffer. Many buffers are known in the art suitable for storing siRNAs. For example, the buffer may be comprised of 100 mM KCl, 30 mM HEPES-pH 7.5, and 1 mM MgCl₂. Preferably, the double stranded polynucleotides of the present invention retain 30% to 100% of their activity when stored in such a buffer at 4° C. for one year. More preferably, they retain 80% to 100% of their biological activity when stored in such a buffer at 4° C. for one year. Alternatively, the compositions can be stored at −20° C. in such a buffer for at least a year or more. Preferably, storage for a year or more at −20° C. results in less than a 50% decrease in biological activity. More preferably, storage for a year or more at −20° C. results in less than a 20% decrease in biological activity after a year or more. Most preferably, storage for a year or more at −20° C. results in less than a 10% decrease in biological activity.

In order to ensure stability of the siRNA pools prior to usage, they may be retained in dried-down form at −20° C. until they are ready for use. Prior to usage, they should be resuspended; however, even once resuspended, for example, in the aforementioned buffer, they should be kept at −20° C. until used. The aforementioned buffer, prior to use, may be stored at approximately 4° C. or room temperature. Effective temperatures at which to conduct transfection are well known to persons skilled in the art, but include for example, room temperature.

Because the ability of the dsRNA of the present invention to retain functionality and to resist degradation is not dependent on the sequence of the bases, the cell type, or the species into which it is introduced, the present invention is applicable across a broad range of organisms, including but not limited plants, animals, protozoa, bacteria, viruses and fungi. The present invention is particularly advantageous for use in mammals such as cattle, horse, goats, pigs, sheep, canines, rodents such as hamsters, mice, and rats, and primates such as, gorillas, chimpanzees, and humans.

The present invention may be used advantageously with diverse cell types include those of the germ cell line, as well as somatic cells. The cells may be stem cells or differentiated cells. For example, the cell types may be embryonic cells, oocytes sperm cells, adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes and cells of the endocrine or exocrine glands.

The present invention is applicable for use for employing RNA interference against a broad range of genes, including but not limited to the 45,000 genes of a human genome, such as those implicated in diseases such as diabetes, Alzheimer's and cancer, as well as all genes in the genomes of the aforementioned organisms.

The polynucleotides of the present invention may be administered to a cell by any method that is now known or that comes to be known and that from reading this disclosure, one skilled in the art would conclude would be useful with the present invention. For example, the polynucleotides may be passively delivered to cells.

Passive uptake of modified polynucleotides can be modulated, for example, by the presence of a conjugate such as a polyethylene glycol moiety or a cholesterol moiety at the 5′ terminal of the sense strand and/or, in appropriate circumstances, a pharmaceutically acceptable carrier.

Preferably, the polynucleotides are double stranded when they are administered.

Other methods include, but are not limited to, transfection techniques employing DEAE-Dextran, calcium phosphate, cationic lipids/liposomes, microinjection, electroporation, immunoporation, and coupling of the polynucleotides to specific conjugates or ligands such as antibodies, antigens, or receptors.

Further, the stabilized dsRNA of the present invention may be used in a diverse set of applications, including but not limited to basic research, drug discovery and development, diagnostics and therapeutics. For example, the present invention may be used to validate whether a gene product is a target for drug discovery or development. In this application, the mRNA that corresponds to a target nucleic acid sequence of interest is identified for targeted degradation. Inventive polynucleotides that are specific for targeting the particular gene are introduced into a cell or organism, preferably in double stranded form. The cell or organism is maintained under conditions allowing for the degradation of the targeted mRNA, resulting in decreased activity or expression of the gene. The extent of any decreased expression or activity of the gene is then measured, along with the effect of such decreased expression or activity, and a determination is made that if expression or activity is decreased, then the nucleic acid sequence of interest is a target for drug discovery or development. In this manner, phenotypically desirable effects can be associated with RNA interference of particular target nucleic acids of interest, and in appropriate cases toxicity and pharmacokinetic studies can be undertaken and therapeutic preparations developed.

The present invention may also be used in RNA interference applications that induce transient or permanent states of disease or disorder in an organism by, for example, attenuating the activity of a target nucleic acid of interest believed to be a cause or factor in the disease or disorder of interest. Increased activity of the target nucleic acid of interest may render the disease or disorder worse, or tend to ameliorate or to cure the disease or disorder of interest, as the case may be. Likewise, decreased activity of the target nucleic acid of interest may cause the disease or disorder, render it worse, or tend to ameliorate or cure it, as the case may be. Target nucleic acids of interest can comprise genomic or chromosomal nucleic acids or extrachromosomal nucleic acids, such as viral nucleic acids.

Further, the present invention may be used in RNA interference applications that determine the function of a target nucleic acid or target nucleic acid sequence of interest. For example, knockdown experiments that reduce or eliminate the activity of a certain target nucleic acid of interest, such as a promoter region in a genome or a structural gene. This can be achieved by performing RNA interference with one or more siRNAs targeting a particular target nucleic acid of interest. Observing the effects of such a knockdown can lead to inferences as to the function of the target nucleic acid of interest. RNA interference can also be used to examine the effects of polymorphisms, such as biallelic polymorphisms, by attenuating the activity of a target nucleic acid of interest having one or the other allele, and observing the effect on the organism or system studied. Therapeutically, one allele or the other, or both, may be selectively silenced using RNA interference where selective allele silencing is desirable.

Still further, the present invention may be used in RNA interference applications, such as diagnostics, prophylactics, and therapeutics. For these applications, an organism suspected of having a disease or disorder that is amenable to modulation by manipulation of a particular target nucleic acid of interest is treated by administering siRNA. Results of the siRNA treatment may be ameliorative, palliative, prophylactic, and/or diagnostic of a particular disease or disorder. Preferably, the siRNA is administered in a pharmaceutically acceptable manner with a pharmaceutically acceptable carrier or diluent.

Therapeutic applications of the present invention can be performed with a variety of therapeutic compositions and methods of administration. Pharmaceutically acceptable carriers and diluents are known to persons skilled in the art. Methods of administration to cells and organisms are also known to persons skilled in the art. Dosing regimens, for example, are known to depend on the severity and degree of responsiveness of the disease or disorder to be treated, with a course of treatment spanning from days to months, or until the desired effect on the disorder or disease state is achieved. Chronic administration of siRNAs may be required for lasting desired effects with some diseases or disorders. Suitable dosing regimens can be determined by, for example, administering varying amounts of one or more siRNAs in a pharmaceutically acceptable carrier or diluent, by a pharmaceutically acceptable delivery route, and amount of drug accumulated in the body of the recipient organism can be determined at various times following administration. Similarly, the desired effect (for example, degree of suppression of expression of a gene product or gene activity) can be measured at various times following administration of the siRNA, and this data can be correlated with other pharmacokinetic data, such as body or organ accumulation. Those of ordinary skill can determine optimum dosages, dosing regimens, and the like. Those of ordinary skill may employ EC₅₀ data from in vivo and in vitro animal models as guides for human studies.

Further, the polynucleotides can be administered in a cream or ointment topically, an oral preparation such as a capsule or tablet or suspension or solution, and the like. The route of administration may be intravenous, intramuscular, dermal, subdermal, cutaneous, subcutaneous, intranasal, oral, rectal, by eye drops, by tissue implantation of a device that releases the siRNA at an advantageous location, such as near an organ or tissue or cell type harboring a target nucleic acid of interest.

Having described the invention with a degree of particularity, examples will now be provided. These examples are not intended to and should not be construed to limit the scope of the claims in any way. Although the invention may be more readily understood through reference to the following examples, they are provided by way of illustration and are not intended to limit the present invention unless specified.

### EXAMPLES

### Example 1

### Synthesizing Polynucleotides

RNA oligonucleotides were synthesized in a stepwise fashion using the nucleotide addition reaction cycle illustrated in FIG. 13. The synthesis is preferably carried out as an automated process on an appropriate machine. Several such synthesizing machines are known to those of skill in the art. Each nucleotide is added sequentially (3′- to 5′-direction) to a solid support-bound oligonucleotide. Although polystyrene supports are preferred, any suitable support can be used. The first nucleoside at the 3′-end of the chain is covalently attached to a solid support. The nucleotide precursor, an activated ribonucleotide such as a phosphoramidite or H-phosphonate, and an activator such as a tetrazole, for example, S-ethyl-tetrazole (although any other suitable activator can be used) are added (step i in FIG. 13), coupling the second base onto the 5′-end of the first nucleoside. The support is washed and any unreacted 5′-hydroxyl groups are capped with an acetylating reagent such as but not limited to acetic anhydride or phenoxyacetic anhydride to yield unreactive 5′-acetyl moieties (step ii). The P(III) linkage is then oxidized to the more stable and ultimately desired P(V) linkage (step iii), using a suitable oxidizing agent such as, for example, t-butyl hydroperoxide or iodine and water. At the end of the nucleotide addition cycle, the 5′-silyl group is cleaved with fluoride ion (step iv), for example, using triethylammonium fluoride or t-butyl ammonium fluoride. The cycle is repeated for each subsequent nucleotide. It should be emphasized that although FIG. 13 illustrates a phosphoramidite having a methyl protecting group, any other suitable group may be used to protect or replace the oxygen of the phosphoramidite moiety. For example, alkyl groups, cyanoethyl groups, or thio derivatives can be employed at this position. Further, the incoming activated nucleoside in step (i) can be a different kind of activated nucleoside, for example, an H-phosphonate, methyl phosphonamidite or a thiophosphoramidite. It should be noted that the initial, or 3′, nucleoside attached to the support can have a different 5′ protecting group such as a dimethoxytrityl group, rather than a silyl group. Cleavage of the dimethoxytrityl group requires acid hydrolysis, as employed in standard DNA synthesis chemistry. Thus, an acid such as dichloroacetic acid (DCA) or trichloroacetic acid (TCA) is employed for this step alone. Apart from the DCA cleavage step, the cycle is repeated as many times as necessary to synthesize the polynucleotide desired.

Following synthesis, the protecting groups on the phosphates, which are depicted as methyl groups in FIG. 13, but need not be limited to methyl groups, are cleaved in 30 minutes utilizing 1 M disodium-2-carbamoyl-2-cyanoethylene-1,1-dithiolate trihydrate (dithiolate) in DMF (dimethylformamide). The deprotection solution is washed from the solid support bound oligonucleotide using water. The support is then treated with 40% methylamine for 20 minutes at 55° C. This releases the RNA oligonucleotides into solution, deprotects the exocyclic amines and removes the acetyl protection on the 2′-ACE groups. The oligonucleotides can be analyzed by anion exchange HPLC at this stage.

The 2′-orthoester groups are the last protecting groups to be removed, if removal is desired. The structure of the 2′-ACE protected RNA immediately prior to 2′-deprotection is as represented in FIG. 14.

For automated procedures, solid supports having the initial nucleoside are installed in the synthesizing instrument. The instrument will contain all the necessary ancillary reagents and monomers needed for synthesis. Reagents are maintained under argon, since some monomers, if not maintained under an inert gas, can hydrolyze. The instrument is primed so as to fill all lines with reagent. A synthesis cycle is designed that defines the delivery of the reagents in the proper order according to the synthesis cycle, delivering the reagents in the order specified in FIG. 13. Once a cycle is defined, the amount of each reagent to be added is defined, the time between steps is defined, and washing steps are defined, synthesis is ready to proceed once the solid support having the initial nucleoside is added.

For the RNA analogs described herein, modification is achieved through three different general methods. The first, which is implemented for carbohydrate and base modifications, as well as for introduction of certain linkers and conjugates, employs modified phosphoramidites in which the modification is pre-existing. An example of such a modification would be the carbohydrate 2′-modified species (2′-F, 2′-NH₂, 2′-O-alkyl, etc.) wherein the 2′ orthoester is replaced with the desired modification 3′ or 5′ terminal modifications could also be introduced such as fluoroscein derivatives, Dabsyl, cholesterol, cyanine derivatives or polyethylene glycol. Certain inter-nucleotide bond modifications would also be introduced via the incoming reactive nucleoside intermediate. Examples of the resultant internucleotide bond modification include but are not limited to methylphosphonates, phosphoramidates, phosphorothioates or phoshorodithioates.

Many modifiers can be employed using the same or similar cycles. Examples in this class would include, for example, 2-aminopurine, 5-methyl cytidine, 5-aminoallyl uridine, diaminopurine, 2-O-alkyl, multi-atom spacers, single monomer spacers, 2′-aminonucleosides, 2′-fluoro nucleosides, 5-iodouridine, 4-thiouridine, acridines, 5-bromouridine, 5-fluorocytidine, 5-fluorouridine, 5-iodouridine, 5-iodocytidine, 5-biotin-thymidine, 5-fluoroscein-thymidine, inosine, pseudouridine, a basic monomer, nebularane, deazanucleoside, pyrene nucleoside, azanucleoside, etc. Often the rest of the steps in the synthesis would remain the same with the exception of modifications that introduce substituents that are labile to standard deprotection conditions. Here modified conditions would be employed that do not effect the substituent. Second, certain internucleotide bond modifications require an alteration of the oxidation step to allow for their introduction. Examples in this class include phosphorothioates and phosphorodithioates wherein oxidation with elemental sulfur or another suitable sulfur transfer agent is required. Third, certain conjugates and modifications are introduced by “post-synthesis” process, wherein the desired molecule is added to the biopolymer after solid phase synthesis is complete. An example of this would be the addition of polyethylene glycol to a pre-synthesized oligonucleotide that contains a primary amine attached to a hydrocarbon linker. Attachment in this case can be achieved by using a N-hydroxy-succinimidyl ester of polyethylene glycol in a solution phase reaction.

While this outlines the most preferred method for synthesis of synthetic RNA and its analogs, any nucleic acid synthesis method which is capable of assembling these molecules could be employed in their assembly. Examples of alternative methods include 5′-DMT-2′-TBDMS and 5′-DMT-2′-TOM synthesis approaches. Some 2′-O-methyl, 2′-F and backbone modifications can be introduced in transcription reactions using modified and wild type T7 and SP6 polymerases, for example.

Synthesizing Modified RNA

The following guidelines are provided for synthesis of modified RNAs, and can readily be adapted to use on any of the automated synthesizers known in the art.

3′ Terminal Modifications

There are several methods for incorporating 3′ modifications. The 3′ modification can be anchored or “loaded” onto a solid support of choice using methods known in the art. Alternatively, the 3′ modification may be available as a phosphoramidite. The phosphoramidite is coupled to a universal support using standard synthesis methods where the universal support provides a hydroxyl at which the 3′ terminal modification is created by introduction of the activated phosphoramidite of the desired terminal modification. Alternatively, the 3′ modification could be introduced post synthetically after the polynucleotide is removed from the solid support. The free polynucleotide initially has a 3′ terminal hydroxyl, amino, thiol, or halogen that reacts with an appropriately activated form of the modification of choice. Examples include but are not limited to N-hydroxy succinimidyl ester, thioether, disulfide, maliemido, or haloalkyl reactions. This modification now becomes the 3′ terminus of the polynucleotide. Examples of modifications that can be conjugated post synthetically can be but are not limited to fluorosceins, acridines, TAMRA, dabsyl, cholesterol, polyethylene glycols, multi-atom spacers, cyanines, lipids, carbohydrates, fatty acids, steroids, peptides, or polypeptides,

5′ Terminal Modifications

There are a number of ways to introduce a 5′ modification into a polynucleotide. For example, a nucleoside having the 5′ modification can be purchased and subsequently activated to a phosphoramidite, for example. The phosphoramidite having the 5′ modification may also be commercially available. Then, the activated nucleoside having the 5′ modification is employed in the cycle just as any other activated nucleoside may be used. However, not all 5′ modifications are available as phosphoramidites. In such an event, the 5′ modification can be introduced in an analogous way to that described for 3′ modifications above.

Thioates

Polynucleotides having one or more thioate moieties, such as phosphorothioate linkages, were made in accordance with the synthesis cycle described above and illustrated in FIG. 13. However, in place of the t-butyl hydroperoxide oxidation step, elemental sulfur or another sulfurizing agent was used.

5′-Thio Modifications

Monomers having 5′ thiols can be purchased as phosphoramidites from commercial suppliers such as Glen Research. These 5′ thiol modified monomers generally bear trityl protecting groups. Following synthesis, the trityl group can be removed by any method known in the art.

Other Modifications

For certain modifications, the steps of the synthesis cycle will vary somewhat. For example, where the 3′ end has an inverse dT (wherein the first base is attached to the solid support through the 5′-hydroxyl and the first coupling is a 3′-3′ linkage) detritylation and coupling occurs more slowly, so extra detritylating reagent, such as dichloroactetic acid (DCA), should be used and coupling time should be increased to 300 seconds. Some 5′ modifications may require extended coupling time. Examples include cholesterol, fluorophores such as Cy3 or Cy5 biotin, dabsyl, amino linkers, thio linkers, spacers, polyethylene glycol, phosphorylating reagent, BODIPY, or photocleavable linkers.

It should be noted that if a polynucleotide is to have only a single modification, that modification can be most efficiently carried out manually by removing the support having the partially built polynucleotide on it, manually coupling the monomer having the modification, and then replacing the support in the automated synthesizer and resuming automated synthesis.

### Example 2

### Deprotection and Cleavage of Synthesized Oligos from the Support

Cleaving can be done manually or in an automated process on a machine. Cleaving of the protecting moiety from the internucleotide linkage, for example a methyl group, can be achieved by using any suitable cleaving agent known in the art, for example, dithiolate or thiophenol. One molar dithiolate in DMF is added to the solid support at room temperature for 10 to 20 minutes. The support is then thoroughly washed with, for example, DMF, then water, then acetonitrile. Alternatively a water wash followed by a thorough acetonitrile will suffice to remove any residual dithioate.

Cleavage of the polynucleotide from the support and removal of exocyclic base protection can be done with 40% aqueous N-methylamine (NMA), followed by heating to 55 degrees Centigrade for twenty minutes. Once the polynucleotide is in solution, the NMA is carefully removed from the solid support. The solution containing the polynucleotide is then dried down to remove the NMA under vacuum. Further processing, including duplexing, desalting, gel purifying, quality control, and the like can be carried out by any method known in the art.

For some modifications, the NMA step may vary. For example, for a 3′ amino modification, the treatment with NMA should be for forty minutes at 55 degrees Centigrade. Puromycin, 5′ terminal amino linker modifications, and 2′ amino nucleoside modifications are heated for 1 hour after addition of 40% NMA. Oligonucleotides modified with Cy5 are treated with ammonium hydroxide for 24 hours while protected from light.

Preparation of Cleave Reagents

HPLC grade water and synthesis grade acetonitrile are used. The dithiolate is pre-prepared as crystals. Add 4.5 grams of dithiolate crystals to 90 mL of DMF. Forty percent NMA can be purchased, ready to use, from a supplier such as Sigma Aldrich Corporation.

Annealing Single Stranded Polynucleotides to Produce Double Stranded siRNA

Single stranded polynucleotides can be annealed by any method known in the art, employing any suitable buffer. For example, equal amounts of each strand can be mixed in a suitable buffer, such as, for example, 50 mM HEPES pH 7.5, 100 mM potassium chloride, 1 mM magnesium chloride. The mixture is heated for one minute at 90 degrees Centigrade, and allowed to cool to room temperature. In another example, each polynucleotide is separately prepared such that each is at 50 micromolar concentration. Thirty microliters of each polynucleotide solution is then added to a tube with 15 microliters of 5× annealing buffer, wherein the annealing buffer final concentration is 100 mM potassium cloride, 30 mM HEPES-KOH pH 7.4 and 2 mM magnesium cloride. Final volume is 75 microliters. The solution is then incubated for one minute at 90 degrees Centigrade, spun in a centrifuge for 15 seconds, and allowed to incubate at 37 degrees Centigrade for one hour, then allowed to come to room temperature. This solution can then be stored frozen at minus 20 degrees Centigrade and freeze thawed up to five times. The final concentration of the duplex is 20 micromolar. An example of a buffer suitable for storage of the polynucleotides is 20 mM KCl, 6 mM HEPES pH 7.5, 0.2 mM MgCl₂. All buffers used should be RNase free.

Removal of the Orthoester Moiety

If desired, the orthoester moiety or moieties may be removed from the polynucleotide by any suitable method known in the art. One such method employs a volatile acetic acid-tetramethylenediamine (TEMED) pH 3.8 buffer system that can be removed by lyophilization following removal of the orthoester moiety or moieties. Deprotection at a pH higher than 3.0 helps minimize the potential for acid-catalyzed cleavage of the phosphodiester backbone. For example, deprotection can be achieved using 100 mM acetic acid adjusted to pH 3.8 with TEMED by suspending the orthoester protected polynucleotide and incubating it for 30 minutes at 60 degrees Centigrade. The solution is then lyophilized or subjected to a SpeedVac to dryness prior to use. If necessary, desalting following deprotection can be performed by any method known in the art, for example, ethanol precipitation or desalting on a reversed phase cartridge.

### Example 3

### Double Stranded Polynucleotides Synthesized for Use in RNA Interference

The following is a list of 19-mer double stranded polynucleotides having a di-dT overhang that were synthesized using Dharmacon, Inc.'s proprietary ACE chemistry, and were designed and used in accordance with the invention described herein. “SEAP” refers to human alkaline phosphatase; “human cyclo” refers to human cyclophylline; an asterisk between nucleotide units refers to a modified internucleotide linkage that is a phosphorothioate linkage; the structure 2′-F—C or 2′-F—U refers to a nucleotide unit having a fluorine atom attached to the 2′ carbon of a ribosyl moiety; the structure 2′-N—C or 2′-N—U refers to a nucleotide unit having an —NH₂ group attached to the 2′ carbon of a ribosyl moiety; the structure 2′-OME-C or 2′-OME-U refers to a nucleotide unit having a 2′-O-methyl modification at the 2′ carbon of a ribosyl moiety; dG, dU, dA, dC, and dT refer to a nucleotide unit that is deoxy with respect to the 2′ position, and instead has a hydrogen attached to the 2′ carbon of the ribosyl moiety. Unless otherwise indicated, all nucleotide units in the list below are ribosyl with an —OH at the 2′ carbon.

| | | |
|---|---|---|
| SP-2217-s | G U G A U G U A U G U C A G A G A G U dT dT | (SEQ. ID. No. 1) |
| SP-2217-as | A C U C U C U G A C A U A C A U C A C dT dT | (SEQ. ID. No. 2) |
| SP-2217-s-p | G U G A U G U A U G U C A G A G A G U dT dT (ACE on) | (SEQ. ID. No. 3) |
| SP-2217-as-p | A C U C U C U G A C A U A C A U C A C dT dT (ACE on) | (SEQ. ID. No. 4) |
| SP-2217-as4 | A C*U*C U C U G A C A U A C A U*C*A C dT dT | (SEQ. ID. No. 5) |
| SP-2217-as8 | A C*U*C*U*C U G A C A U A C*A*U*C*A C dT dT | (SEQ. ID. No. 6) |
| SP-2217-as8F | A 2′-F-C*2′-F-U*2′-F-C*2′-F-U*2′-F-C 2′-F-U G A 2′-F-C A 2′-F-U A | (SEQ. ID. No. 7) |
| 2′-F-C*A*2′-F-U*2′-F-C*A 2′-F-C dT dT | | |
| SP-s-N | G 2′-N-U G A 2′-N-U G 2′-N-U A 2′-N-U G 2′-N-U 2′-N-C A G A G A | (SEQ. ID. No. 8) |
| G 2′-N-U dT dT | | |
| SP-as-N-12 | A 2′-N-C 2′-N-U 2′-N-C 2′-N-U 2′-N-C U G A C A 2′-N-U A 2′-N-C A | (SEQ. ID. No. 9) |
| 2′-N-U 2′-N-C A 2′-N-C dT dT | | |
| SP-s-thio | G*U*G*A*U*G*U*A*U*G*U*C*A*G*A*G*A*G*U dT dT | (SEQ. ID. No. 10) |
| SP-as-thio | A*C*U*C*U*C*U*G*A*C*A*U*A*C*A*U*C*A*C dT dT | (SEQ. ID. No. 11) |
| SP-as-thio12 | A*C*U*C*U*C*U G A C A U A*C*A*U*C*A*C dT dT | (SEQ. ID. No. 12) |
| SP-s-M | G 2′-OME-U G A 2′-OME-U G 2′-OME-U A 2′-OME-U G 2′-OME-U | (SEQ. ID. No. 13) |
| 2′-OME-C A G A G A G 2′-OME-U dT dT | | |
| SP-as | | |
| SP-as-M10 | A 2′-OME-C 2′-OME-U 2′-OME-C 2′-OME-U 2′-OME-C U G A C A | (SEQ. ID. No. 14) |
| 2′-OME-U A 2′-OME-C A 2′-OME-U 2′-OME-C A 2′-OME-C dT dT | | |
| SP-2217-s | dG U dG A dU G dU A dU G dU C dA G dA G dA G dU dT dT | (SEQ. ID. No. 15) |
| SP-2217-as | A dC U dC U dC U G A C A U A dC A dU C dA C dT dT | (SEQ. ID. No. 16) |

Human Cyclo

| H-cyclo-476-s | |
|---|---|
| UGGUGUUUGGCAAAGUUCU dT dT | (SEQ. ID. No. |
| | 17) |
| H-cyclo-476-as | |
| AGAACUUUGCCAAACACCA dT dT | (SEQ. ID. No. |
| | 18) |
| H-cyc-f-s | |
| (2′-F-U) G G (2′-F-U) G (2′-F-U) (2′-F-U) (2′-F-U) G G (2′-F-C) A A A G (2′-F-U) (2′-F- | (SEQ. ID. No. |
| U) (2′-F-C) (2′-F-U) dT dT | 19) |
| H-cyc-f-as9 | |
| A G A A (2′-F-C) (2′-F-U) (2′-F-U) (2′-F-U) G (2′-F-C) (2′-F-C) A A A (2′-F-C) A (2′-F- | (SEQ. ID. No. |
| C) (2′-F-C) A dT dT | 20) |
| H-cyc-f-as8 | |
| A G A A (2′-F-C) (2′-F-U) (2′-F-U) U G (2′-F-C) (2′-F-C) A A A (2′-F-C) A (2′-F-C) (2′- | (SEQ. ID. No. |
| F-C) A dT dT | 21) |
| H-cyclo-476-as6 | |
| A G A A (2′-F-C) (2′-F-U) (2′-F-U) U G C C A A A (2′-F-C) A (2′-F-C) (2′-F-C) A dT | (SEQ. ID. No. |
| dT | 22) |
| H-cyclo-476-as1 | |
| A G A A C U U (2′-F-U) G C C A A A C A C C A dT dT | (SEQ. ID. No. |
| | 23) |

Firefly Luciferase

| | |
|---|---|
| Luc-1188-2′F-s | |
| G A 2′F-U 2′F-U A 2′F-U G 2′F-U 2′F-C 2′F-C G G 2′F-U 2′F-U A 2′F-U G 2′F-U A | (SEQ. ID. No. 24) |
| dT dT | |
| Luc-1188-2′F-as | |
| 2′F-U A 2′F-C A 2′F-U A A 2′F-C 2′F-C G G A 2′F-C A 2′F-U A A 2′F-U 2′F-C dT | (SEQ. ID. No. 25) |
| dT | |
| Fluc-s-d1 | |
| dG A U U A U G U C C G G U U A U G U A dT dT | (SEQ. ID. No. 26) |
| Fluc-s-d2 | |
| G dA U U A U G U C C G G U U A U G U A dT dT | (SEQ. ID. No. 27) |
| Fluc-s-d3 | |
| G A dU U A U G U C C G G U U A U G U A dT dT | (SEQ. ID. No. 28) |
| Fluc-s-d4 | |
| G A U dU A U G U C C G G U U A U G U A dT dT | (SEQ. ID. No. 29) |
| Fluc-s-d5 | |
| G A U U dA U G U C C G G U U A U G U A dT dT | (SEQ. ID. No. 30) |
| Fluc-s-d6 | |
| G A U U A dU G U C C G G U U A U G U A dT dT | (SEQ. ID. No. 31) |
| Fluc-s-d7 | |
| G A U U A U dG U C C G G U U A U G U A dT dT | (SEQ. ID. No. 32) |
| Fluc-s-d8 | |
| G A U U A U G dU C C G G U U A U G U A dT dT | (SEQ. ID. No. 33) |
| Fluc-s-d9 | |
| G A U U A U G U dC C G G U U A U G U A dT dT | (SEQ. ID. No. 34) |
| Fluc-s-d10 | |
| G A U U A U G U C dC G G U U A U G U A dT dT | (SEQ. ID. No. 35) |
| Fluc-s-d11 | |
| G A U U A U G U C C dG G U U A U G U A dT dT | (SEQ. ID. No. 36) |
| Fluc-s-d12 | |
| G A U U A U G U C C G dG U U A U G U A dT dT | (SEQ. ID. No. 37) |
| Fluc-s-d13 | |
| G A U U A U G U C C G G dU U A U G U A dT dT | (SEQ. ID. No. 38) |
| Fluc-s-d14 | |
| G A U U A U G U C C G G U dU A U G U A dT dT | (SEQ. ID. No. 39) |
| Fluc-s-d15 | |
| G A U U A U G U C C G G U U dA U G U A dT dT | (SEQ. ID. No. 40) |
| Fluc-s-d16 | |
| G A U U A U G U C C G G U U A dU G U A dT dT | (SEQ. ID. No. 41) |
| Fluc-s-d17 | |
| G A U U A U G U C C G G U U A U dG U A dT dT | (SEQ. ID. No. 42) |
| Fluc-s-d18 | |
| G A U U A U G U C C G G U U A U G dU A dT dT | (SEQ. ID. No. 43) |
| Fluc-s-d19 | |
| G A U U A U G U C C G G U U A U G U dA dT dT | (SEQ. ID. No. 44) |
| Fluc-as-d1 | |
| dU A C A U A A C C G G A C A U A A U C dT dT | (SEQ. ID. No. 45) |
| Fluc-as-d2 | |
| U dA C A U A A C C G G A C A U A A U C dT dT | (SEQ. ID. No. 46) |
| Fluc-as-d3 | |
| U A dC A U A A C C G G A C A U A A U C dT dT | (SEQ. ID. No. 47) |
| Fluc-as-d4 | |
| U A C dA U A A C C G G A C A U A A U C dT dT | (SEQ. ID. No. 48) |
| Fluc-as-d5 | |
| U A C A dU A A C C G G A C A U A A U C dT dT | (SEQ. ID. No. 49) |
| Fluc-as-d6 | |
| U A C A U dA A C C G G A C A U A A U C dT dT | (SEQ. ID. No. 50) |
| Fluc-as-d7 | |
| U A C A U A dA C C G G A C A U A A U C dT dT | (SEQ. ID. No. 51) |
| Fluc-as-d8 | |
| U A C A U A A dC C G G A C A U A A U C dT dT | (SEQ. ID. No. 52) |
| Fluc-as-d9 | |
| U A C A U A A C dC G G A C A U A A U C dT dT | (SEQ. ID. No. 53) |
| Fluc-as-d10 | |
| U A C A U A A C C dG G A C A U A A U C dT dT | (SEQ. ID. No. 54) |
| Fluc-as-d11 | |
| U A C A U A A C C G dG A C A U A A U C dT dT | (SEQ. ID. No. 55) |
| Fluc-as-d12 | |
| U A C A U A A C C G G dA C A U A A U C dT dT | (SEQ. ID. No. 56) |
| Fluc-as-d13 | |
| U A C A U A A C C G G A dC A U A A U C dT dT | (SEQ. ID. No. 57) |
| Fluc-as-d14 | |
| U A C A U A A C C G G A C dA U A A U C dT dT | (SEQ. ID. No. 58) |
| Fluc-as-d15 | |
| U A C A U A A C C G G A C A dU A A U C dT dT | (SEQ. ID. No. 59) |
| Fluc-as-d16 | |
| U A C A U A A C C G G A C A U dA A U C dT dT | (SEQ. ID. No. 60) |
| Fluc-as-d17 | |
| U A C A U A A C C G G A C A U A dA U C dT dT | (SEQ. ID. No. 61) |
| Fluc-as-d18 | |
| U A C A U A A C C G G A C A U A A dU C dT dT | (SEQ. ID. No. 62) |
| Fluc-as-d19 | |
| U A C A U A A C C G G A C A U A A U dC dT dT | (SEQ. ID. No. 63) |

### Example 4

### Performing RNA Interference

### Transfection

SiRNA duplexes were annealed using standard buffer (50 millimolar HEPES pH 7.5, 100 millimolar KCl, 1 mM MgCl₂). The transfections are done according to the standard protocol described below.

Standard Transfection Protocol for 96 Well and 6 Well Plates: siRNAs

1. Protocols for 293 and Calu6, HeLas, MDA 75 are identical.

2. Cell are plated to be 95% confluent on the day of transfection.

3. SuperRNAsin (Ambion) is added to transfection mixture for protection against RNAses.

4. All solutions and handling have to be carried out in RNAse free conditions.

Plate 10.5-1 ml in 25 ml of media in a small flask or 1 ml in 50 ml in a big flask.

96 Well Plate

1. Add 3 ml of 0.05% trypsin-EDTA in a medium flask (6 in a bid) incubate 5 min at 37 degrees C.

2. Add 7 ml (14 ml big) of regular media and pipet 10 times back and forth to re-suspend cells.

3. Take 25 microliters of the cell suspension from step 2 and 75 microliters of trypan blue stain (1:4) and place 10 microliters in a cell counter.

4. Count number of cells in a squares with 3 lines walls.

5. Average number of cells×4×10000 is number of cells per ml.

6. Dilute with regular media to have 350 000/ml .

7. Plate 100 microliters (35000 cell for HEK293) in a 96 well plate.

Transfection. For 2×96 Well Plates (60 Well Format)

1. OPTI-MEM 2 ml+80 microliters Lipofectamine 2000 (1:25)+15 microliters of SuperRNAsin (AMBION)

2. Transfer iRNA aliquots (0.8 microliters of 100 micromolar to screen (total dilution factor is 1:750, 0.8 microliters of 100 micromolar solution will give 100 nanomolar final) to the dipdish in a desired order (Usually 3 columns×6 for 60 well format or four columns by 8 for 96 well)

3. Transfer 100 microliters of OPTI-MEM

4. Transfer 100 microliters of OPTI-MEM with Lipofectamine 2000 and SuperRNAsin to each well

5. Leave for 20-30 min RT

6. Add 0.55 ml of regular media to each well. Cover plate with film and mix.

7. Array out 100×3×2 directly to the cells (sufficient for two plates).

Transfection. For 2×6 Well Plates

8. 8 ml OPTI-MEM+160 microliters Lipofectamine 2000 (1:25). 30 microliters of SuperRNAsin (AMBION)

9. Transfer iRNA aliquots (total dilution factor is 1:750, 5 microliters of 100 micromolar solution will give 100 nanomolar final) to polystyrene tubes.

10. Transfer 1300 microliters of OPTI-MEM with Lipofectamine 2000 and SuperRNAsin (AMBION)

11. Leave for 20-30 min RT

12. Add 0.55 ml of regular media to each well. Cover plate with film and mix.

13. Transfer 2 ml to each well (sufficient for two wells)

The mRNA or protein levels are measured 24, 48, 72, and 96 hours post transfection with standard kits or Custom B-DNA sets and Quantigene kits (Bayer).

### Example 5

### Measurement of Activity/Detection

The level of siRNA-induced RNA interference, or gene silencing, was estimated by assaying the reduction in target mRNA levels or reduction in the corresponding protein levels. Assays of mRNA levels were carried out using B-DNA™ technology (Quantagene Corp.). Protein levels for fLUC and rLUC were assayed by STEADY GLO™ kits (Promega Corp.). Human alkaline phosphatase levels were assayed by Great EscAPe SEAP Fluorescence Detection Kits (#K2043-1), BD Biosciences, Clontech.

Although the invention has been described and has been illustrated in connection with certain specific or preferred inventive embodiments, it will be understood by those of skill in the art that the invention is capable of many further modifications. This application is intended to cover any and all variations, uses, or adaptations of the invention that follow, in general, the principles of the invention and include departures from the disclosure that come within known or customary practice within the art and as may be applied to the essential features described in this application and in the scope of the appended claims.

What is claimed is:

1. A method of performing RNA interference, said method comprising exposing a double stranded polynucleotide to a target nucleic acid, wherein said double stranded polynucleotide is comprised of a sense strand and an antisense strand, and wherein at least one of said sense strand and said antisense strand comprises at least one orthoester modified nucleotide.

2. The method according to claim 1, wherein said at least one orthoester modified nucleotide is located on said sense strand.

3. The method according to claim 2, wherein the antisense strand comprises at least one nucleotide selected from the group consisting of a 2′ halogen modified nucleotide, a 2′ amine modified nucleotide, a 2′-O-alkyl modified nucleotide and a 2′ alkyl modified nucleotide.

4. The method according to claim 3, wherein the antisense strand comprises at least one 2′ halogen modified nucleotide and said halogen is fluorine.

5. The method according to claim 2, wherein the double stranded polynucleotide further comprises a conjugate.

6. The method according to claim 5, wherein said conjugate is selected from the group consisting of amino acids, peptides, polypeptides, proteins, sugars, carbohydrates, lipids, polymers, nucleotides, polynucleotides, and combinations thereof.

7. The method according to claim 5, wherein the conjugate is cholesterol.

8. The method according to claim 5, wherein conjugate is polyethylene glycol.

9. The method according to claim 1, wherein the double stranded polynucleotide comprises 18-30 nucleotide base pairs.

10. The method according to claim 9, wherein the double stranded polynucleotide comprises 19 nucleotide base pairs.

11. The method according to claim 1, wherein the double stranded polynucleotide has an overhang of at least one nucleotide unit on at least one of said sense strand and said antisense strand.

12. The method according to claim 1, wherein at least one strand of the double stranded polynucleotide comprises at least one modified internucleotide linkage.

13. The method according to claim 12, wherein the modified internucleotide linkage is selected from the group consisting of a phosphorothioate linkage and a phosphorodithioate linkage.

14. The method according to claim 1, wherein at least one strand of the double stranded polynucleotide is a polyribonucleotide.

15. A method of performing RNA interference, said method comprising exposing a double stranded polynucleotide to a target nucleic acid, wherein said double stranded polynucleotide is comprised of:
(i) a sense strand,
(ii) an antisense strand, and
(iii) a conjugate,
wherein at least one of said sense strand and said antisense strand comprises a 2′ modified nucleotide.

16. A double stranded polynucleotide comprising:
(a) a sense strand, wherein said sense strand comprises a polynucleotide that is comprised of at least one orthoester modified nucleotide; and
(b) an antisense strand, wherein said antisense strand comprises a polynucleotide that is comprised of at least one 2′ modified nucleotide.

17. The double stranded polynucleotide of claim 16, wherein the antisense strand comprises at least one nucleotide selected from the group consisting of a 2′ halogen modified nucleotide, a 2′ amine modified nucleotide, a 2′-O-alkyl modified nucleotide and a 2′ alkyl modified nucleotide.

18. The double stranded polynucleotide of claim 17, wherein the 2′ modified nucleotide is a 2′ halogen modified nucleotide and said halogen is fluorine.

19. The double stranded polynucleotide of claim 16, further comprising a conjugate.

20. The double stranded polynucleotide of claim 19, wherein said conjugate is selected from the group consisting of amino acids, peptides, polypeptides, proteins, sugars, carbohydrates, lipids, polymers, nucleotides, polynucleotides, and combinations thereof.

21. The double stranded polynucleotide of claim 19, wherein said conjugate is cholesterol.

22. The double stranded polynucleotide of claim 19, wherein said conjugate is polyethylene glycol.

23. The double stranded polynucleotide of claim 16, wherein said double stranded polynucleotide is comprised of 18-30 nucleotide base pairs.

24. The double stranded polynucleotide of claim 23, wherein said double stranded polynucleotide is comprised of 19 nucleotide base pairs.

25. The double stranded polynucleotide of claim 16, further comprising an overhang of at least one nucleotide unit on at least one of said sense strand and said antisense strand.

26. The double stranded polynucleotide of claim 16, wherein at least one of said sense strand and said antisense strand comprises at least one modified internucleotide linkage.

27. The double stranded polynucleotide of claim 26, wherein the modified internucleotide linkage is selected from the group consisting of a phosphorothoate linkage and a phosphorodithioate linkage.

28. The double stranded polynucleotide of claim 16, wherein at least one of said sense strand and said antisense strand is a polyribonucleotide.

29. A double stranded polynucleotide comprising:
(a) a sense strand, wherein said sense strand comprises a polynucleotide that is comprised of at least one orthoester modified nucleotide;
(b) an antisense strand, wherein said antisense strand comprises a polynucleotide that is comprised of at least one 2′ modified nucleotide; and
(c) a conjugate.

30. The double stranded polynucleotide of claim 29, wherein the conjugate is located on the sense strand.

31. The double stranded polynucleotide of claim 29, wherein the conjugate is located on the antisense strand.

32. The double stranded polynucleotide of claim 29, wherein the antisense strand comprises at least one nucleotide selected from the group consisting of a 2′ halogen modified nucleotide, a 2′ amine modified nucleotide, a 2′-O-alkyl modified nucleotide and a 2′ alkyl modified nucleotide.

33. The double stranded polynucleotide of claim 32, wherein the sense strand is comprised of a 2′ halogen modified nucleotide and said halogen is fluorine.

34. The double stranded polynucleotide of claim 29, wherein the conjugate is selected from the group consisting of amino acids, peptides, polypeptides, proteins, sugars, carbohydrates, lipids, polymers, nucleotides, polynucleotides, and combinations thereof.

35. The double stranded polynucleotide of claim 29, wherein the conjugate is cholesterol.

36. The double stranded polynucleotide of claim 29, wherein the conjugate is polyethylene glycol.

37. The double stranded polynucleotide of claim 29, wherein said polynucleotide is comprised of 18-30 nucleotide base pairs.

38. The double stranded polynucleotide of claim 37, wherein said polynucleotide is comprised of 19 nucleotide base pairs.

39. The double stranded polynucleotide of claim 29, further comprising an overhang of at least one nucleotide unit on at least one of said sense strand and said antisense strand.

40. The double stranded polynucleotide of claim 29, wherein at least one of said sense strand and said antisense strand comprises at least one modified internucleotide linkage.

41. The double stranded polynucleotide of claim 40, wherein the modified internucleotide linkage is selected from the group consisting of a phosphorothioate linkage and a phosphorodithioate linkage.

42. The double stranded polynucleotide of claim 29, wherein at least one of said sense strand and said antisense strand is a polyribonucleotide.

43. A double stranded polynucleotide comprising:
(a) a sense strand comprised of at least one orthoester modified nucleotide;
(b) an antisense strand; and
(c) a conjugate.

44. The double stranded polynucleotide of claim 43, wherein said conjugate is located on the sense strand.

45. The double stranded polynucleotide of claim 43, wherein said is located on the antisense strand.

46. The double stranded polynucleotide of claim 43 wherein the antisense strand comprises at least one nucleotide selected from the group consisting of a 2′ halogen modified nucleotide, a 2′ amine modified nucleotide, a 2′-O-alkyl modified nucleotide and a 2′ alkyl modified nucleotide.

47. The double stranded polynucleotide of claim 46, wherein the antisense strand is comprised of a 2′ halogen modified nucleotide and said halogen is fluorine.

48. The double stranded polynucleotide of claim 43, wherein the conjugate is selected from the group consisting of amino acids, peptides, polypeptides, proteins, sugars, carbohydrates, lipids, polymers, nucleotides, polynucleotides, and combinations thereof.

49. The double stranded polynucleotide of claim 43, wherein the conjugate is cholesterol.

50. The double stranded polynucleotide of claim 43, wherein the conjugate is polyethylene glycol.

51. The double stranded polynucleotide of claim 43, wherein the polynucleotide is comprised of 18-30 nucleotide base pairs.

52. The double stranded polynucleotide of claim 51, wherein the polynucleotide is comprised of 19 nucleotide base pairs.

53. The double stranded polynucleotide of claim 43, further comprising an overhang of at least one nucleotide unit on at least one of said sense stand and said antisense strand.

54. The double stranded polynucleotide of claim 43, wherein at least one of said sense strand and said antisense strand comprises at least one modified internucleotide linkage.

55. The double stranded polynucleotide of claim 43, wherein at least one of said sense strand and said antisense strand is a polyribonucleotide.

56. A double stranded polynucleotide comprising:
(a) a sense strand;
(b) an antisense strand; and
(c) a conjugate;
wherein the sense strand and/or the antisense strand comprises at least one 2′ modified nucleotide.

57. The double stranded polynucleotide of claim 56, wherein the 2′ modified nucleotide is selected from the group consisting of a 2′ halogen modified nucleotide, a 2′ amine modified nucleotide, a 2′-O-alkyl modified nucleotide and a 2′ alkyl modified nucleotide.

58. The double stranded polynucleotide of claim 57, wherein the 2′ modified nucleotide is a 2′ halogen modified nucleotide and said halogen is fluorine.

59. The double stranded polynucleotide of claim 56, wherein the conjugate is selected from the group consisting of amino acids, peptides, polypeptides, proteins, sugars, carbohydrates, lipids, polymers, nucleotides, polynucleotides, and combinations thereof.

60. The double stranded polynucleotide of claim 56, wherein the conjugate is cholesterol.

61. The double stranded polynucleotide of claim 56, wherein the conjugate is polyethylene glycol.

62. The double stranded polynucleotide of claim 56, wherein said polynucleotide is comprised of 18-30 nucleotide base pairs.

63. The double stranded polynucleotide of claim 56, wherein said polynucleotide is comprised of 19 nucleotide base pairs.

64. The double stranded polynucleotide of claim 56, further comprising an overhang of at least one nucleotide unit on at least one of said sense strand and said antisense strand.

65. The double stranded polynucleotide of claim 56, wherein at least one of said sense strand and said antisense strand comprises at least one modified internucleotide linkage.

66. The double stranded polynucleotide of claim 65, wherein the modified internucleotide linkage is selected from the group consisting of a phosphorothioate linkage and a phosphorodithioate linkage.

67. The double stranded polynucleotide of claim 56, wherein at least one of said sense strand and said antisense strand is a polyribonucleotide.

68. A double stranded polyribonucleotide comprising:
(a) a sense strand, wherein said sense strand is comprised of at least one 2′ orthoester modified nucleotide;
(b) an antisense strand, wherein said antisense strand is comprised of at least one 2′ modified nucleotide selected from the group consisting of a 2′ halogen modified nucleotide, a 2′ amine modified nucleotide, a 2′-O-alkyl modified nucleotide, and a 2′ alkyl modified nucleotide; and
(c) a conjugate selected from the group consisting of amino acids, peptides, polypeptides, proteins, sugars, carbohydrates, lipids, polymers, nucleotides, polynucleotides, and combinations thereof;
wherein said polyribonucleotide comprises between 18 and 30 nucleotide base pairs.

69. A composition comprising: 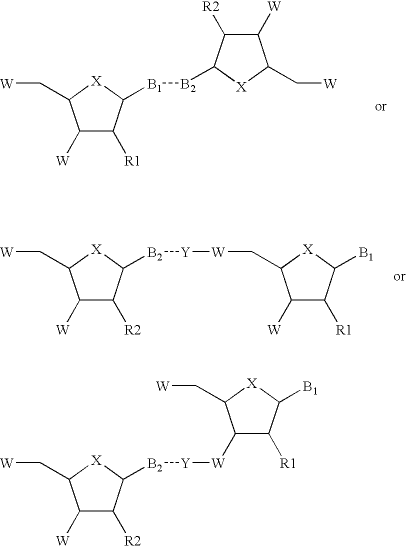
wherein:
each of B₁ and B₂ is a nitrogenous base, heterocycle or carbocycle;
X is selected from the group consisting of O, S, C, and N;
W is selected from the group consisting of an OH, a phosphate, a phosphate ester, a phosphodiester, a phosphotriester, a modified internucleotide link, a conjugate, a nucleotide, and a polynucleotide;
R1 is an orthoester;
R2 is selected from the group consisting of a 2′-O-alkyl group, an alkyl group, and amine, and a halogen; and
Y is a nucleotide or polynucleotide.
